SEQUENCE LISTING (5 sequences)

SEQ ID NO: 1 - protein, 46 aa, synthetic construct
FKAPNAPKRPGGAAYRAKGKPDGGPAEARLTSALGGALAAVLEELR

SEQ ID NO: 2 - protein, 10 aa, synthetic construct
FKDPNAPKRP

SEQ ID NO: 3 - protein, 10 aa, synthetic construct
AAYRAKGKPD

SEQ ID NO: 4 - protein, 10 aa, synthetic construct
PAEARLTSAL

SEQ ID NO: 5 - protein, 10 aa, synthetic construct
ALAAVLEELR

(19) United States
(12) Patent Application Publication
(10) Pub. No.: US 2026/0159546 A1
(43) Pub. Date: Jun. 11, 2026

(54) PHARMACEUTICAL COMPOSITION FOR TREATING SEPSIS INCLUDING DUAL ALARMIN-RECEPTOR-SPECIFIC TARGETING PEPTIDE

(71) Applicant: Industry-University Cooperation Foundation Hanyang University Erica Campus, Ansan-si, (KR)

(72) Inventors: Chul-Su Yang, Ansan-si, (KR); Seong Jae Kim, Ansan-si (KR); Seok-Jun Mun, Ansan-si (KR); Euni Cho, Ansan-si (KR)

(21) Appl. No.: 19/178,915

(22) Filed: Apr. 15, 2025

(30) Foreign Application Priority Data
May 28, 2024 (KR) 10-2024-0069528

(51) Int. Cl. C07K 7/06; A61K 9/127; A61K 38/00; A61K 47/69; A61P 31/04

(52) U.S. Cl. CPC C07K 7/06; A61K 9/127; A61K 47/6911; A61P 31/04; A61K 38/00; C07K 2319/00

(57) ABSTRACT

The present invention relates to a novel alarmin/PRR-targeting system using a TLR4/MD2/RAGE-targeting peptide (TMR peptide) derived from HMGB1, PTX3-TLR4/MD2, or RAGE-interacting domains. The TMR peptide was further functionalized by conjugation with a liposomal delivery system (TMR-Lipo) to improve its unfavorable pharmacokinetic properties. It was demonstrated through the present invention that the TMR-Lipo suppressed TLR4- and RAGE-mediated inflammation by blocking the alarmin-PRR axis in LPS-treated macrophages. Most importantly, the combination of TMR-lipo and antibiotics, which possess both immunomodulatory and antimicrobial capacities, significantly affected the survival of CLP-induced septic mice by targeting the alarmin-PRR axis, thereby mitigating exacerbated inflammation. This synergistic therapeutic approach involving antibiotics and TMR-lipo has great promise as a therapeutic strategy for sepsis.

### CROSS-REFERENCE TO RELATED APPLICATION

This application claims priority to and the benefit of Korean Patent Application No. 2024-0069528, filed on May 28, 2024, the disclosure of which is incorporated herein by reference in its entirety.

### INCORPORATION OF SEQUENCE LISTING

This application contains a sequence listing submitted in Computer Readable Form (CRF). The CRF file contains the sequence listing entitled “9-PK004027457-SequenceListing.xml”, which was created on Apr. 15, 2025, and is 5,577 bytes in size. The information in the sequence listing is incorporated herein by reference in its entirety.

### BACKGROUND

### 1. Field of the Invention

The present invention relates to a pharmaceutical composition for treating sepsis, including a dual alarmin receptor-specific targeting peptide.

### 2. Discussion of Related Art

Sepsis is a clinical syndrome characterized by systemic inflammation caused by infection and is associated with substantial mortality (in-hospital mortality rates 30% to 50%). In 2016, the Third International Consensus Definitions for Sepsis and Septic Shock (Sepsis-3) defined sepsis as “organ dysfunction resulting from a dysregulated host immune response to infection,” marking the first acknowledgement of the vital role of the innate and adaptive immune responses in sepsis pathophysiology. Sepsis pathophysiology is characterized by a hyperinflammatory phase together with systemic activation of the innate immune system. The initial phase of sepsis involves host recognition of microbial pathogen-associated molecular patterns (PAMPs), such as endotoxins, or endogenous damage-associated molecular patterns (DAMPs), which are identified by pattern recognition receptors (PRRs) predominantly expressed on the surface of immune cells.

This recognition initiates host intracellular signaling that converges toward early activation genes such as interferon regulatory factors (IRFs) and nuclear factor-κB (NF-κB). Overall, this systemic activation of the innate immune system by PAMPs and DAMPs sustains an inflammatory response, which is characterized by the uncontrolled release of inflammatory cytokines including tumor necrosis factor alpha (TNF-α), interleukin-1 (IL-1), IL-6, and IL-10, known as the “cytokine storm.” This leads to an increased risk of diseases such as organ dysfunction and septic shock. Therapeutic interventions including cytokine and receptor blockade have shown promise in preclinical settings but have shown disappointing results in clinical trials.

For example, clinical trials have shown that TNF antagonists are the most effective in chronic inflammatory diseases such as rheumatoid arthritis, but have shown limited efficacy in patients with sepsis. This may be because these cytokines serve as “early proinflammatory mediators” in the pathogenesis of sepsis. Indeed, the release of TNF-α occurs within several minutes after lipopolysaccharide (LPS) exposure, making timely intervention in the treatment of sepsis too difficult. Therefore, therapeutic approaches targeting potential late mediators of sepsis may be beneficial for patients with sepsis.

Recent findings that alarmins are pivotal mediators of sepsis pathophysiology provide valuable insights into the modulation of septic responses. High-mobility group protein B1 (HMGB1) and pentraxin 3 (PTX3), the best characterized alarmins, are late mediators of endotoxemia and sepsis, which are rapidly released into the blood by damaged organs and tissue.

These alarmins serve as innate amplifiers of infection through alarmin-PRR interactions, and their release is accompanied by the upregulation of their cognate receptors. For example, it was found that the toll-like receptor 4 (TLR4)/myeloid differentiation factor 2 (MD-2) complex and receptor for advanced glycation end products (RAGE) promote innate inflammatory responses by interacting with these alarmins in septic shock, and their inhibition offers a protective effect in polymicrobial infection-based sepsis models.

In addition, these alarmins appear to serve as late mediators of sepsis, offering a clinically relevant period for pharmacological intervention by targeting the alarmin-receptor axis. Therefore, interrupting the inflammatory feedback loop resulting from an abnormally increased alarmin/PRR axis suppresses inflammation and restores a physiologically appropriate immune response to infection.

### RELATED ART DOCUMENTS

### Non-Patent Document

(Non-Patent Document 1) H. E. Harris, U. Andersson, D. S. Pisetsky, HMGB1: a multifunctional alarmin driving autoimmune and inflammatory disease, Nat Rev Rheumatol, 8 (2012) 195-202.

### SUMMARY OF THE INVENTION

The present invention is directed to providing a fusion peptide for treating sepsis.

The present invention relates to a fusion peptide in which four peptides consisting of amino acid sequences represented by SEQ ID NO: 2 to SEQ ID NO: 5 are connected by a linker. In one embodiment of the present invention, the linker is (G)ₙ, (A)ₙ, or (G₄S)ₘ, wherein m is an integer from 1 to 3, n is an integer from 2 to 8, and in one embodiment of the present invention, the fusion peptide includes an amino acid sequence represented by (SEQ ID NO: 2)-(linker)-(SEQ ID NO: 3)-(linker)-(SEQ ID NO: 4)-(linker)-(SEQ ID NO: 5).

The present invention also provides a fusion peptide including an amino acid sequence represented by SEQ ID NO: 1.

The present invention provides a pharmaceutical composition for treating sepsis, including a fusion peptide in which four peptides consisting of amino acid sequences represented by SEQ ID NO: 2 to SEQ ID NO: 5 are connected by a linker or a fusion peptide including an amino acid sequence represented by SEQ ID NO: 1. In one embodiment of the present invention, the fusion peptide is encapsulated in a liposome, and in another embodiment of the present invention, the fusion peptide is bound to a membrane of the liposome.

The present invention provides a nucleic acid molecule encoding a fusion peptide in which four peptides consisting of amino acid sequences represented by SEQ ID NO: 2 to SEQ ID NO: 5 are connected by a linker or a fusion peptide including an amino acid sequence represented by SEQ ID NO: 1.

One embodiment of the present invention provides a recombinant vector including the nucleic acid molecule.

One embodiment of the present invention provides a recombinant strain including the nucleic acid molecule.

One embodiment of the present invention provides a health functional food for preventing or alleviating sepsis, including the fusion peptide.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

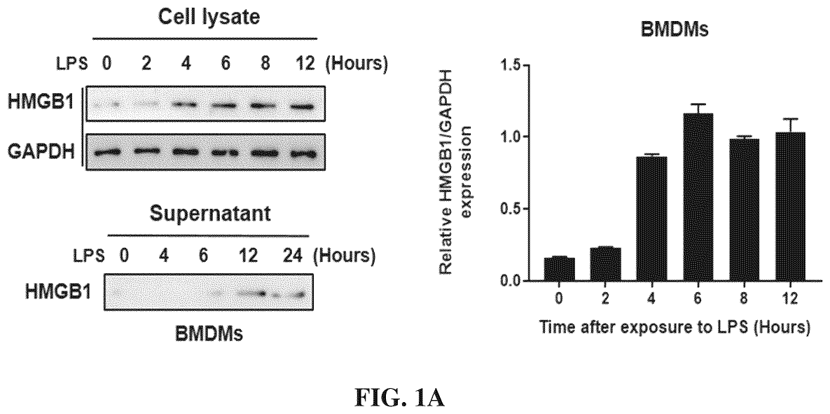

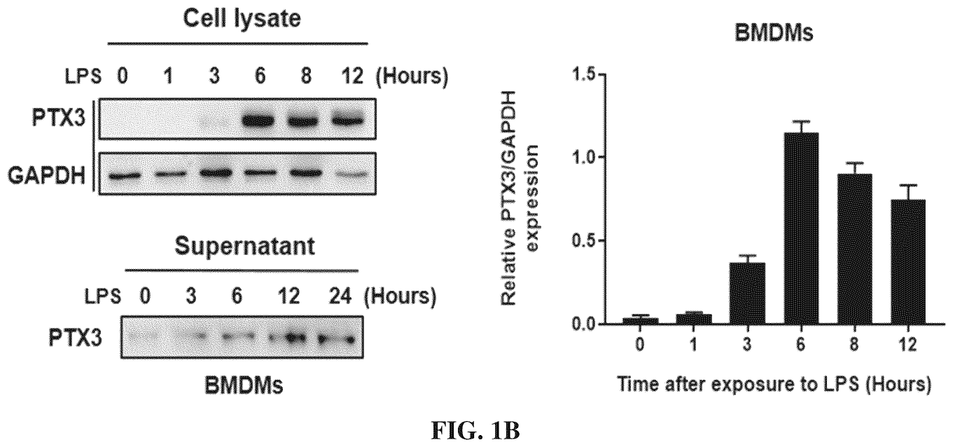

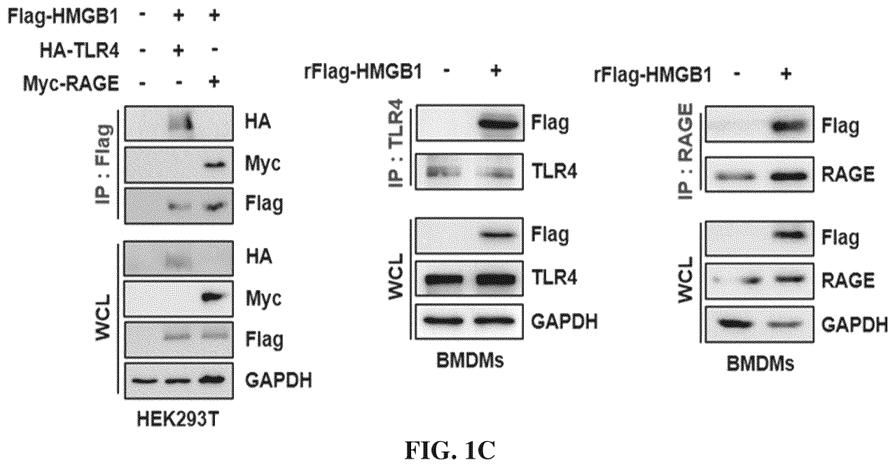

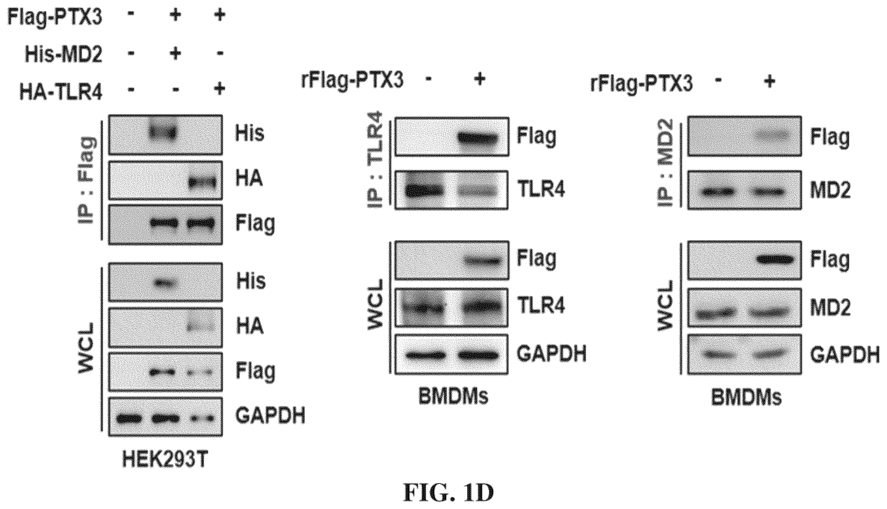

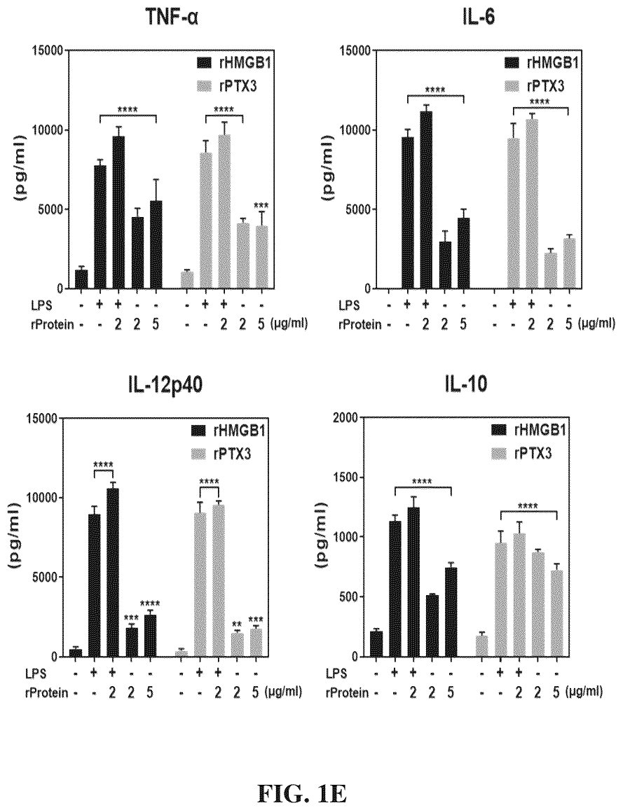

FIG. 1. Interaction of extracellular high-mobility group protein B1 (HMGB1) and (pentraxin 3) PTX3 with toll-like receptor 4 (TLR4) or receptor for advanced glycation end products (RAGE) in murine macrophages;
(A and B) Release of HMGB1 (A) or PTX3 (B) from murine macrophages after stimulation with lipopolysaccharide (LPS). LPS-induced HMGB1 (A) or PTX3 (B) release from murine macrophages. Immunoblots (IB) for HMGB1 and PTX3 in the cell lysates in LPS-treated bone marrow-derived macrophages (BMDMs) and the supernatant at indicated time points after LPS (100 ng/ml);
(C and D) Interaction between HMGB1 (C) or PTX3 (D) with receptors thereof. HEK293T cells were transfected with constructs, and cell lysates were immunoprecipitated with a Flag-tag-specific antibody and analyzed by immunoblotting (IB) with the indicated antibodies. BMDMs were incubated with 10 μg/mL rFlag-HMGB1 (C) or rFlag-PTX3 (D) for three hours, and cell lysates were immunoprecipitated with αTLR4 or αRAGE (C, right), and αTLR4 or alpha-myeloid differentiation factor 2 (αMD-2) (D, right). Immunoprecipitants and whole cel lysates (WCL) were analyzed by immunoblotting with the indicated antibodies;
(E) TNF-α, IL-6, IL-12p40, and IL-10 levels in the supernatant of LPS (100 ng/ml, 18 h) or recombinant alarmins (rHMGB1 or rPTX3, each (2, 5 μg/mL))-treated BMDMs determined by enzyme-linked immunosorbent assay (ELISA). The data shown represents at least three independent experiments (n≥3), and bars denote mean±SEM (****p<0.001), and significance was measured by two-way analysis of variance (ANOVA);

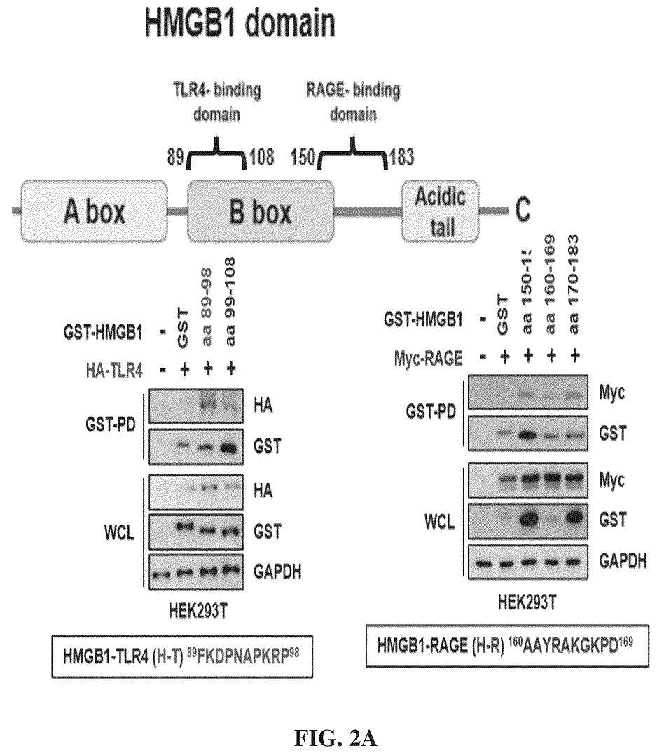

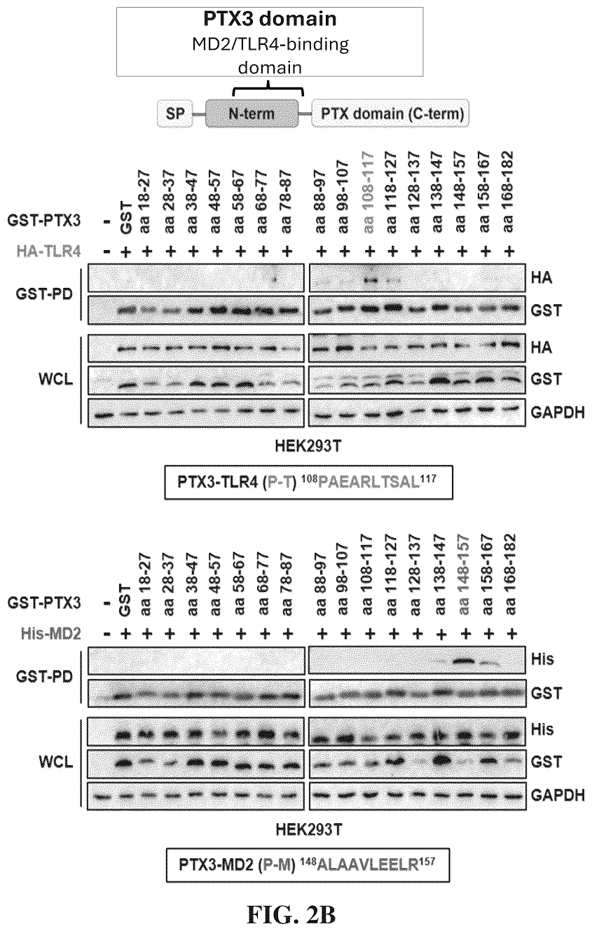

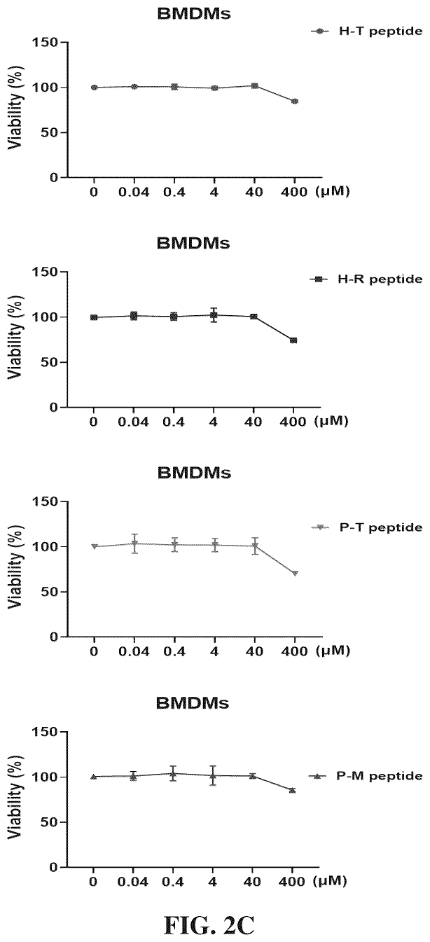

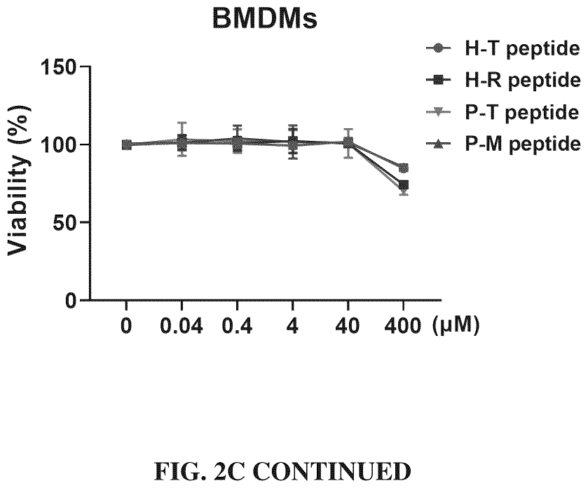

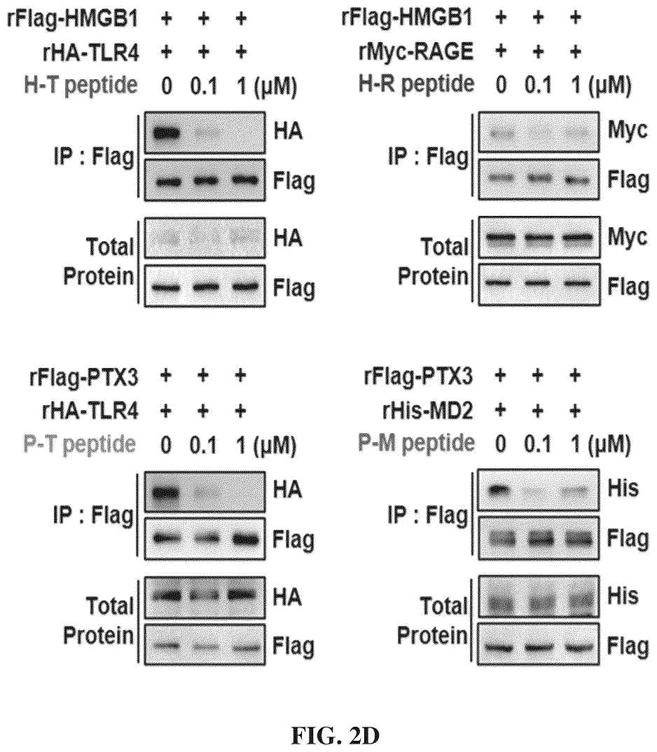

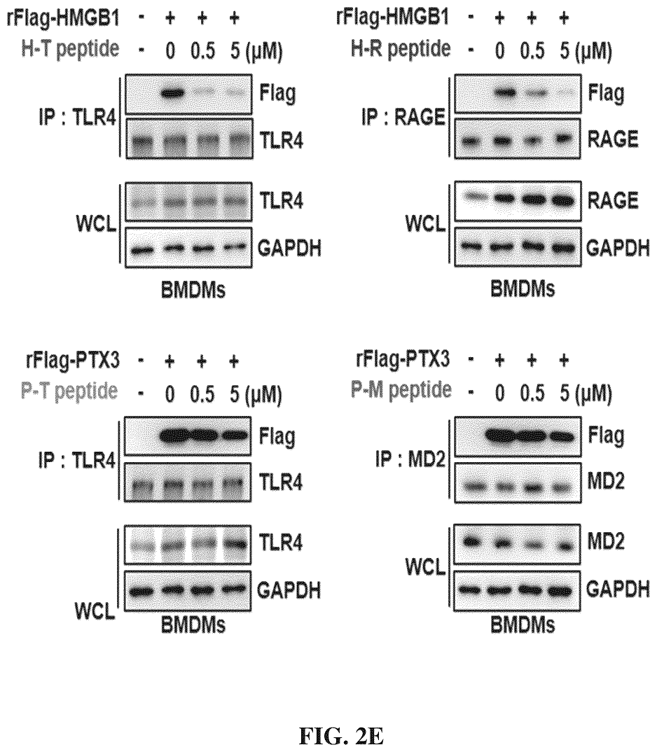

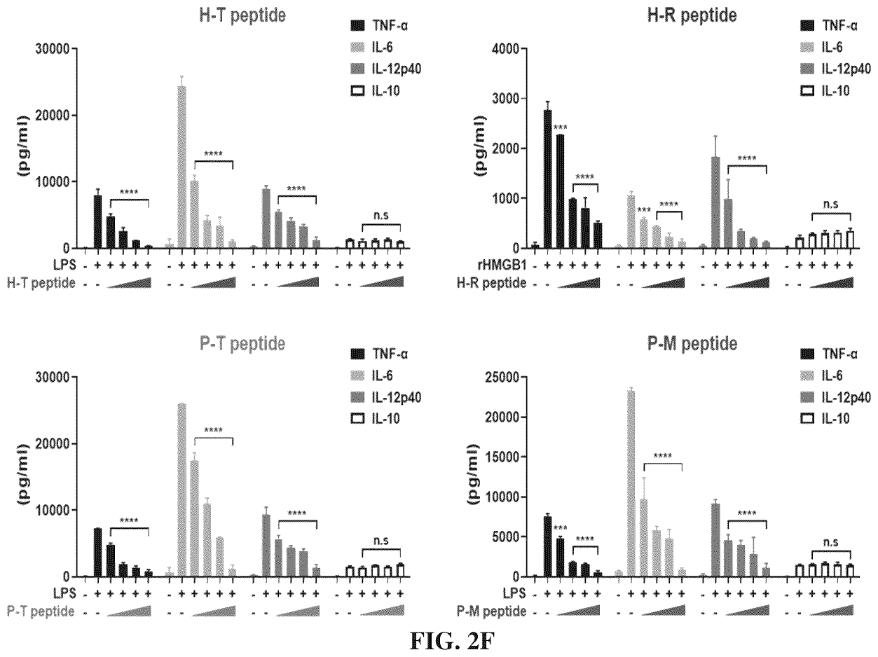

FIG. 2. Preparation of TLR4/MD2/RAGE-interacting motifs of HMGB1 or PTX3;
(A and B) Schematic diagram of HMGB1 (A) and PTX3 (B) domains. Glutathione s-transferase (GST)-tagged truncates including the TLR4-, RAGE-, or MD2-binding domains of HMGB1 or PTX3 were constructed. The bottom panel (A and B) shows the GST-pulldown of the confirmed tagged receptors (HA-TLR4, Myc-RAGE, and His-MD2) with partially expressed GST-tagged truncates. HEK293T cells transfected with the GST-tagged constructs in combination with HA-TLR4, Myc-RAGE or His-MD2 were confirmed by GST-pulldown. Western blots of WCLs and eluates after GST-pulldown are shown, using glyceraldehyde 3-phosphate dehydrogenase (GAPDH) as a loading control in WCLs. Based on the pulldown binding results, TLR4-, MD2-, and RAGE-targeted peptides were constructed and prepared as an H-T, H-R, P-T, or P-M peptide (indicated in panels A and B);
(C) BMDMs were incubated with the H-T, H-R, P-T, or P-M peptide at the indicated concentrations for 48 h, and then cell viability was measured by a cell viability assay. The data shown is the mean±SD of three experiments;
(D) Cell-free immunoprecipitation (IP) assay using the H-T, H-R, P-T, or P-M peptide. 5 μg alarmin protein (rHMGB1 or rPTX3) was mixed with 5 μg of each indicated tagged receptor protein (HA-TLR4, Myc-RAGE, and His-MD2), and treated for four hours as indicated to confirm the competitive inhibitory effect of peptides. They were immunoprecipitated with αFlag for two hours, separated by sodium dodecyl sulfate-polyacrylamide gel electrophoresis (SDS-PAGE), and evaluated by immunoblotting;
(E) Competition between rHMGB1 (top) or PTX3 (bottom) and the constructed peptides for their respective receptors. After treatment with rHMGB1 (top) or PTX3 (bottom) (10 μg/mL, 3 h) together with increasing amounts of the indicated peptides, IP was performed using BMDMs, IB was performed using αTLR4 or αRAGE;
(F) Decrease of LPS-induced cytokine production and rHMGB1 cytokine production induced by the H-T, P-T, P-M, or H-R peptide. TNF-α, IL-6, IL-12p40, and IL-10 levels in the supernatant of the indicated peptide-treated BMDMs, along with LPS (100 ng/mL, 18 h), were determined by ELISA. The data shown represents at least three independent experiments (n≥3), bars denote mean±SEM (***p<0.001, ****p<0.001), and significance was measured by two-way ANOVA. n.s=not significant;

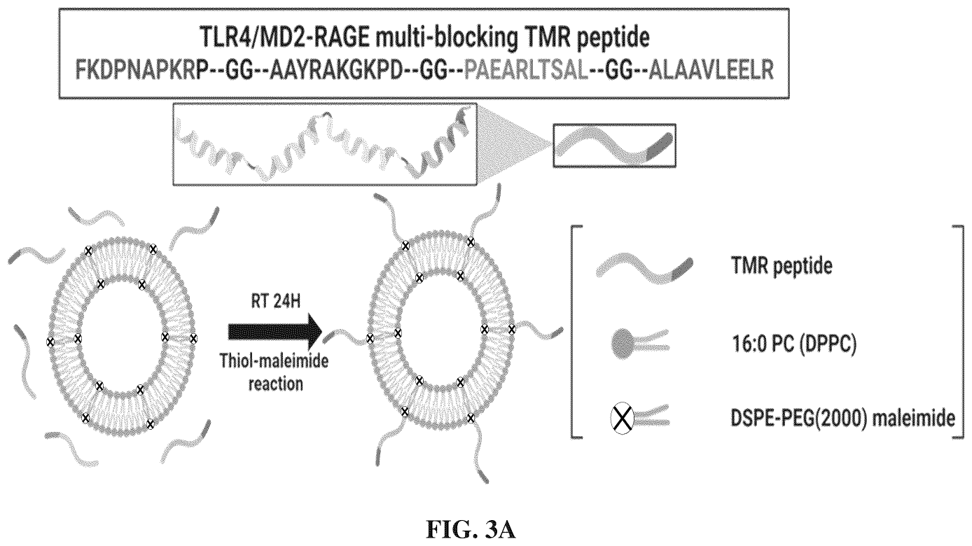

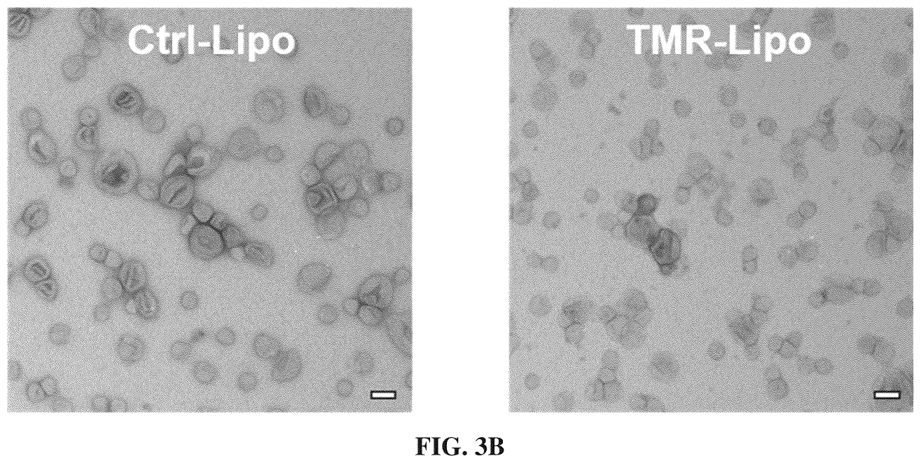

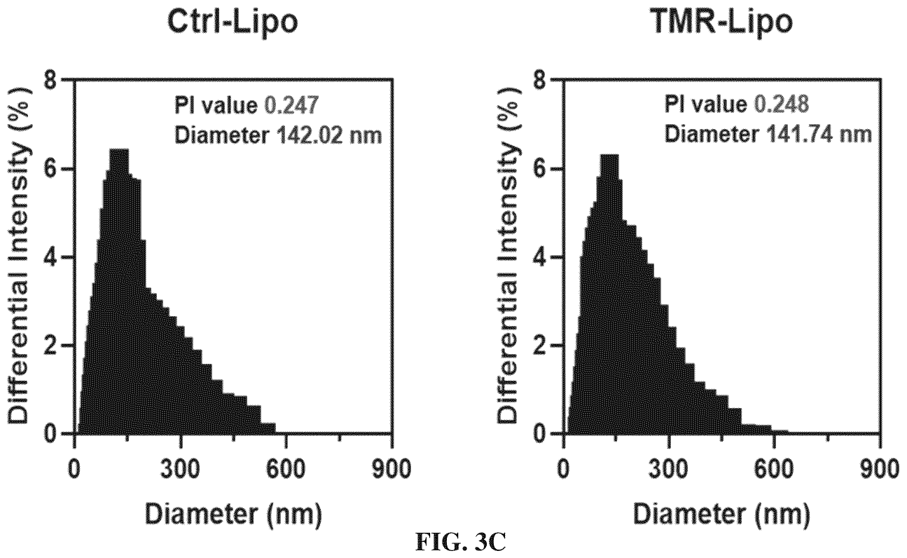

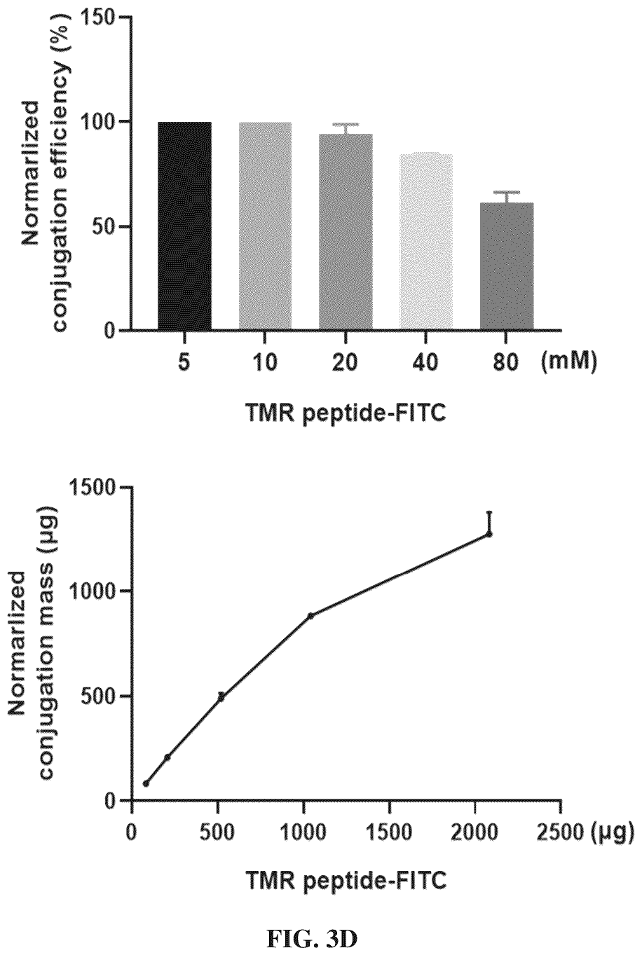

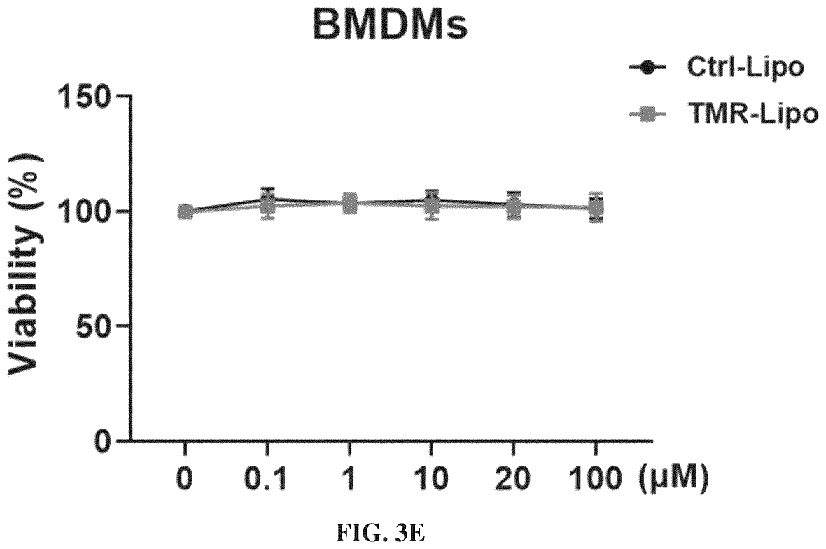

FIG. 3. Preparation and characterization of TLR4/MD2/RAGE-blocking peptide-conjugated liposomes (TMR-Lipo);
(A) Schematic design of TMR peptide (top) and preparation of TMR-conjugated liposomes (bottom);
(B) Transmission electron microscopy (TEM) images of control liposome (Ctrl-Lipo) and TMR-Lipo. Scale bars are 100 nm;
(C) Hydrodynamic particle size of Ctrl-Lipo and TMR-Lipo;
(D) Normalized conjugation efficiency of TMR peptide in TMR-Lipo;
(E) In vitro cell viability of Ctrl-Lipo and TMR-Lipo evaluated for BMDMs. The data shown is the mean±SD of three experiments;

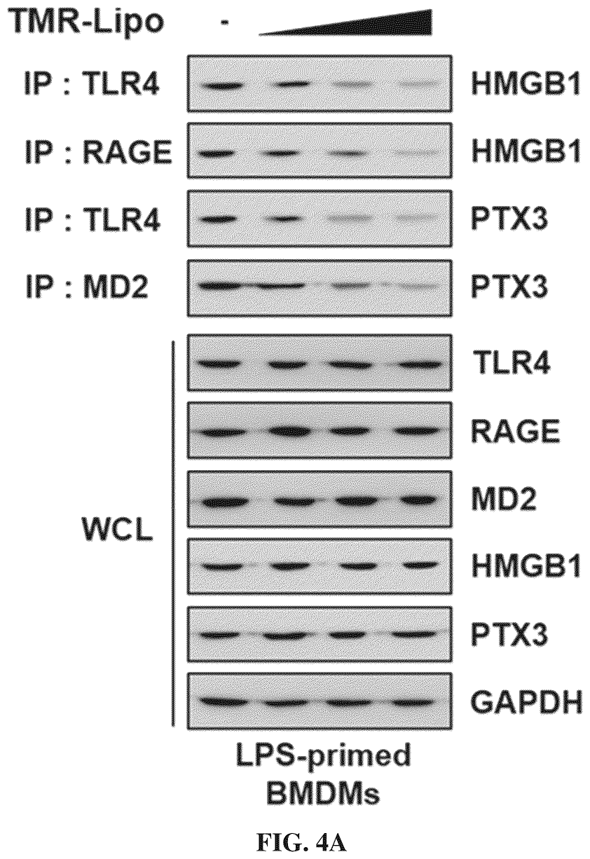

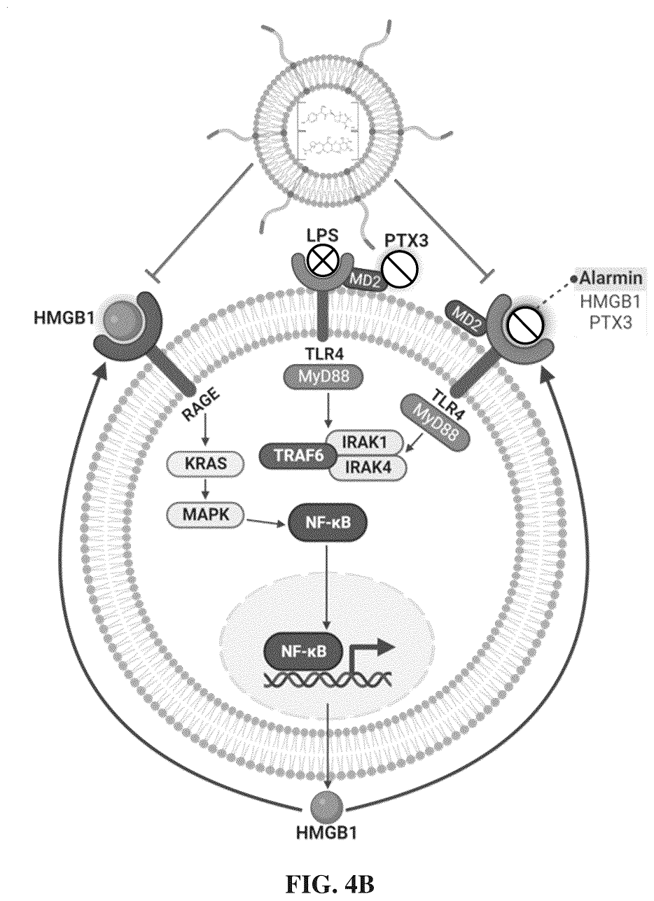

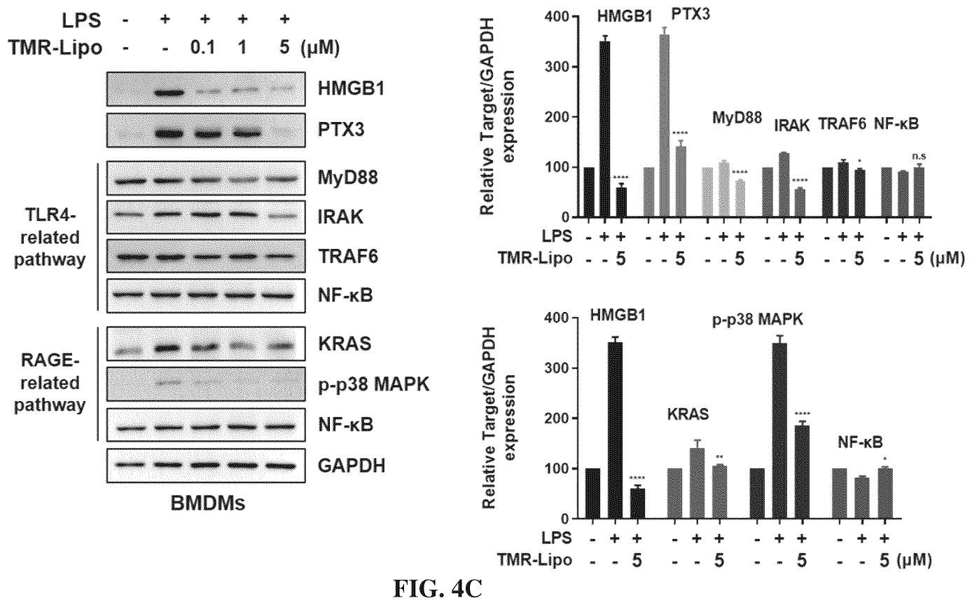

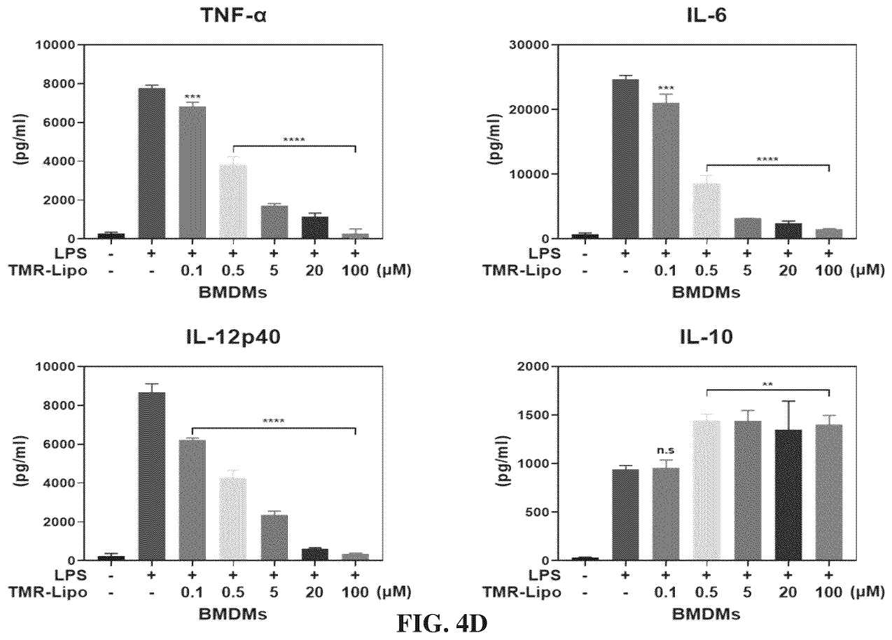

FIG. 4. Inhibitory effects of TMR-Lipo on TLR4/RAGE-mediated responses;
(A) LPS-primed BMDMs (primed with 100 ng/ml LPS for 12 h) were treated with TMR-Lipo (0.1, 1, 5 μM) for 12 h, and then co-IP was performed with αTLR4, αRAGE, or αMD2 and IB was performed with the indicated antibodies;
(B) Schematic representation of TLR4 and RAGE signaling pathways and therapeutic targeting of TMR-Lipo;
(C) BMDMs were treated with TMR-Lipo at the indicated concentrations (2 h), followed by treatment with LPS (100 ng/mL, 8 h). The amounts of HMGB1, PTX3, MyD88, IRAK, TRAF6, NF-κB, KRAS, and p-p38 were measured by IB in the WCL. GAPDH was used as a control. The data shown represents at least three independent experiments (n≥3), and bars denote mean±SEM (**p<0.01, ****p<0.001), and significance was measured by two-way ANOVA. n.s=not significant;
(D) BMDMs were treated with TMR-Lipo (0.1, 0.5, 5, 20, or 100 μM) in the presence LPS for 18 h. The secretion levels of TNF-α, IL-6, IL-12p40, and IL-10 were measured by ELISA. The data shown represents at least three independent experimental replicates (n≥3), and bars denote mean±SEM (**p<0.01, ****p<0.001), and significance was measured by two-way ANOVA. n.s=not significant; and

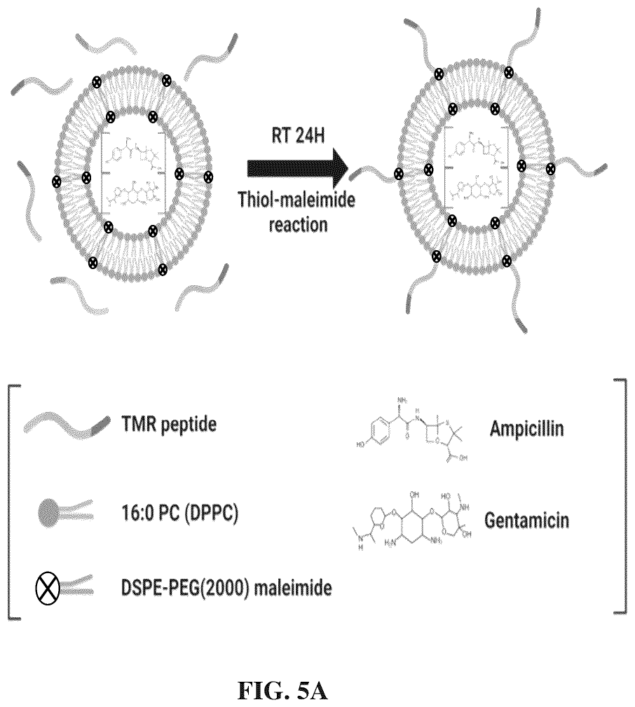

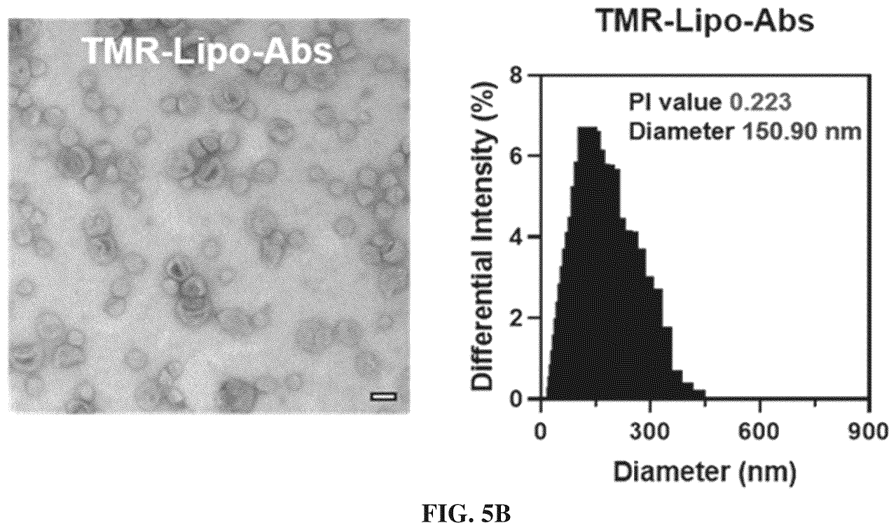

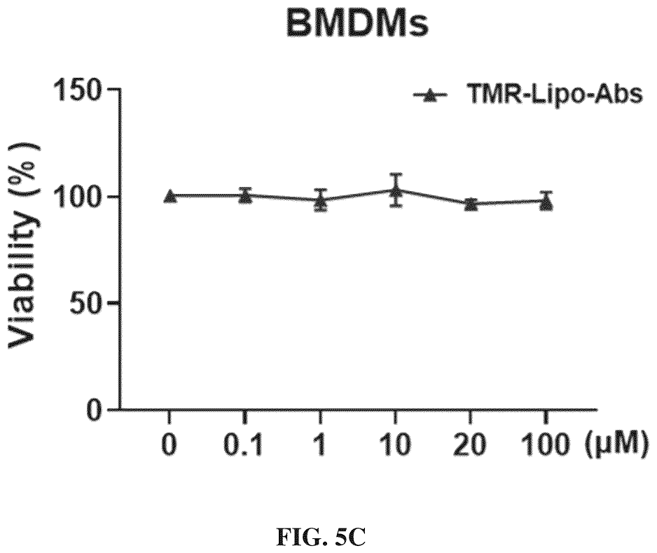

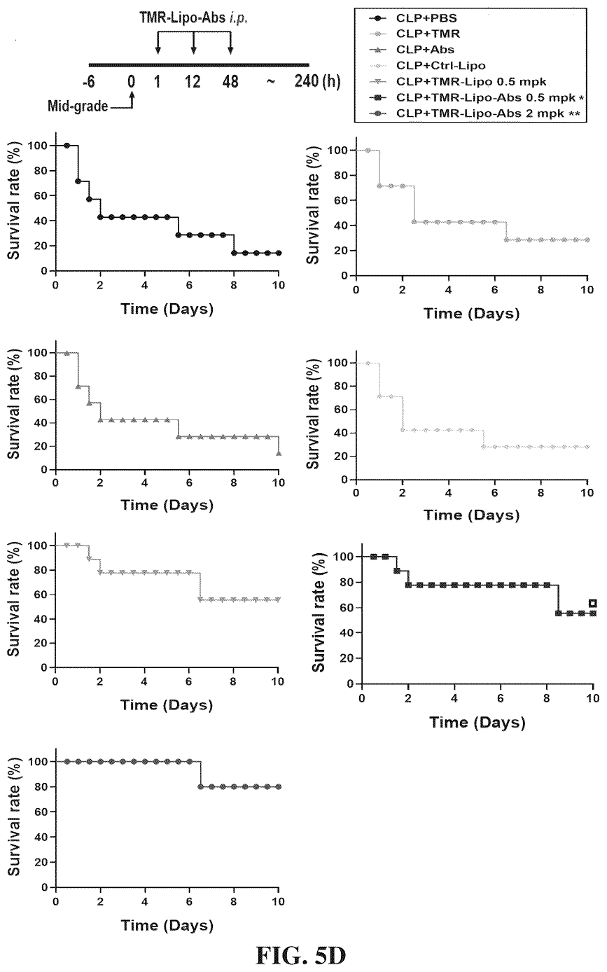

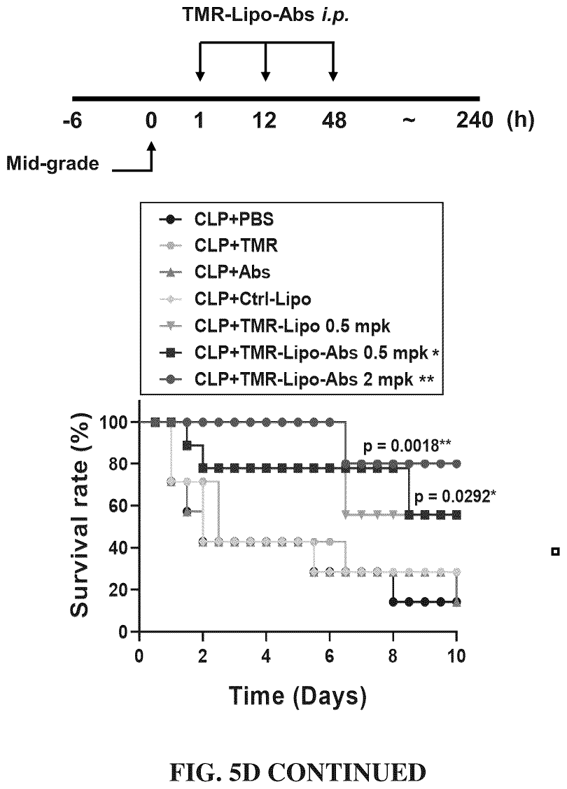

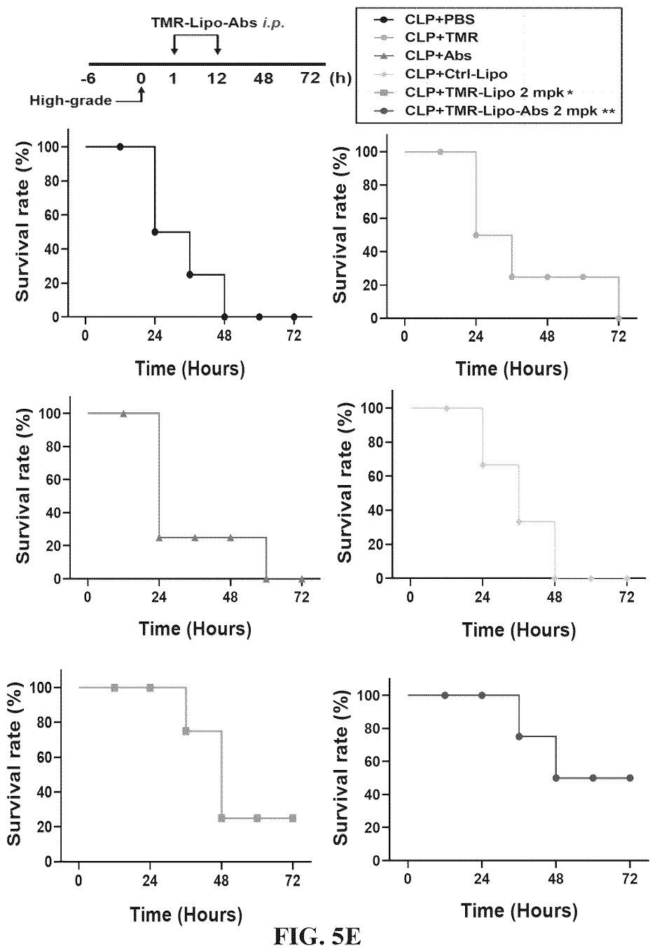

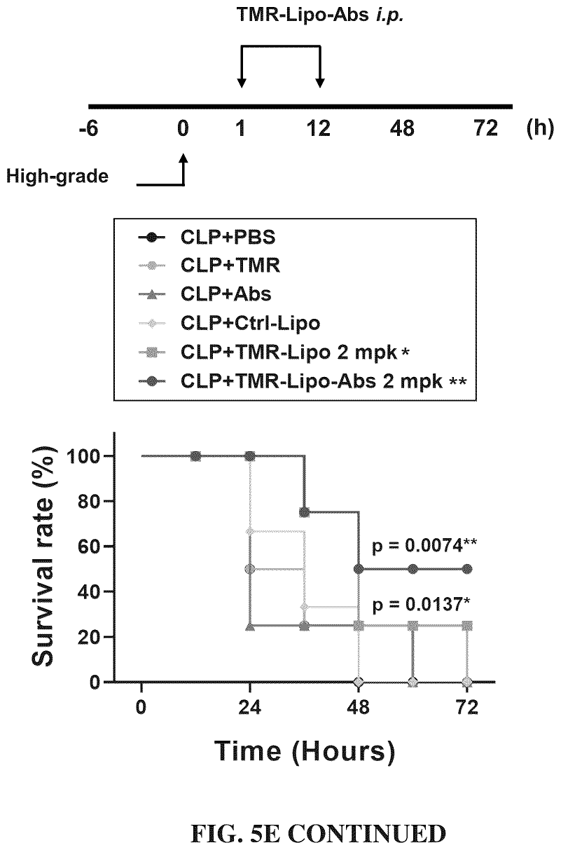

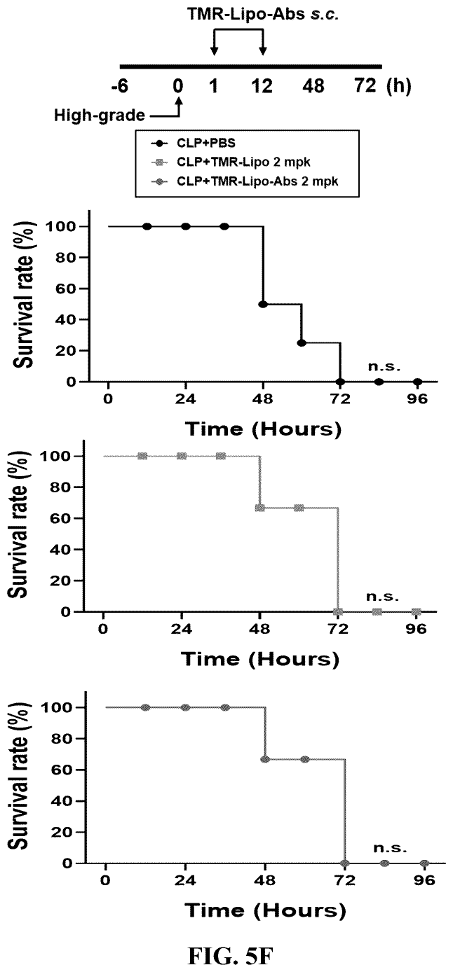

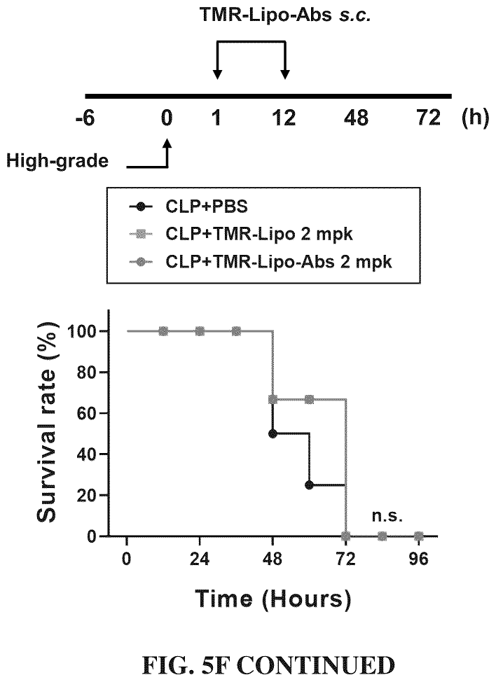

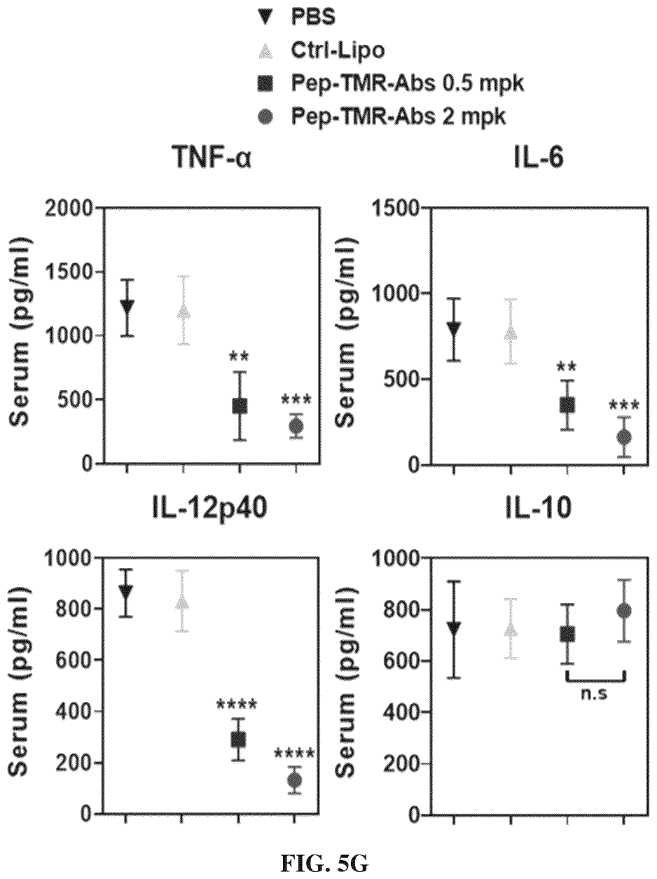

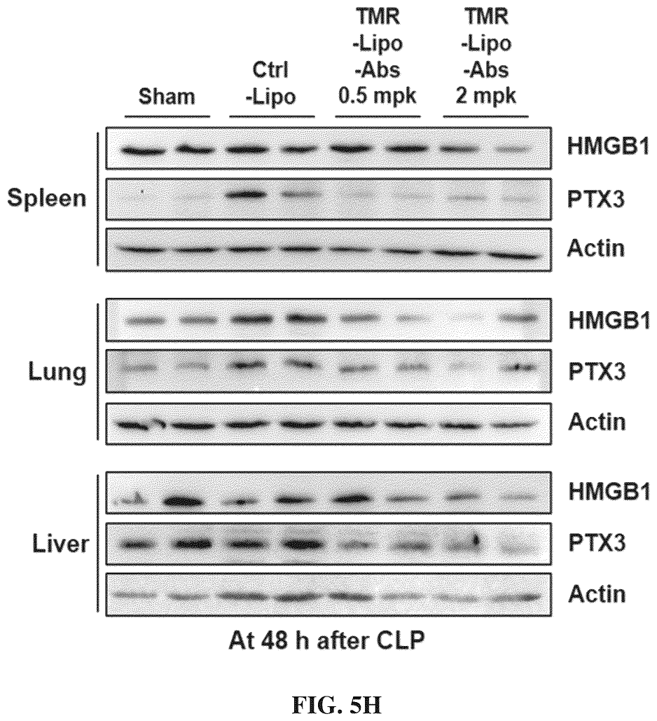

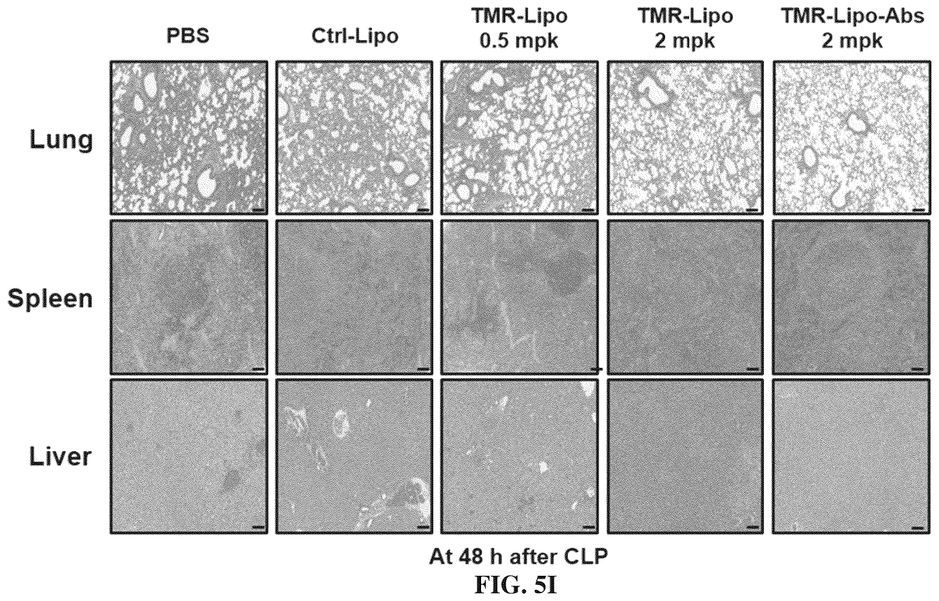

FIG. 5. Antibiotic-loaded TMR-Lipo (TMR-Lipo-Abs) protects mice from CLP-induced polymicrobial sepsis;
(A to C) Preparation and characterization of TMR-Lipo-Abs;
(A) Schematic design of TMR-Lipo-Abs;
(B) TEM image (left) of hydrodynamic particle size (right) of TMR-Lipo-Abs. Scale bar is 100 nm;
(C) In vitro cytotoxicity evaluation of TMR-Lipo-Abs on BMDMs;
(D to F) Schematic design of mid-grade (D) or high-grade (E and F) sepsis mouse model induced by cecal ligation and puncture (CLP) depending on the position of the cecal ligation (top). The survival of CLP mice treated with the indicated therapy was monitored for 10 days (D) or 72 h (E and F). The mortality was measured for n=10 mice per group (bottom). Statistical differences compared with phosphate buffered saline (PBS) or TMR-Lipo-Abs-treated mice are indicated (log-rank test). The data represents three independent experimental replicates with similar results;
(G) Serum cytokine levels. The data shown represents at least three independent experimental replicates (n≥3), and significance was measured by ordinary one-way ANOVA (TNF-α, **p=0.0010 and ***p=0.0002, IL-6, **p=0.0057 and ***p=0.0004 and IL12p40, both ****p<0.0001). n.s=not significant;
(H) IB identification and comparison of HMGB1 and PTX3 expression in spleen, lung, and liver cells of CLP mice after different treatments as damage-associated molecular patterns (DAMP) indicators for tissue damage; and
(I) Representative hematoxylin and eosin (H&E) staining of the lung, spleen, and liver from 3 mice per group. Scale bar is 100 μm.

### DETAILED DESCRIPTION OF EXEMPLARY EMBODIMENTS

Hereinafter, the present invention will be described in detail through embodiments of the present invention with reference to the attached drawings. However, the following embodiments are provided as examples of the present invention, and when it is judged that a detailed description of a technology or configuration well known to those skilled in the art may unnecessarily obscure the gist of the present invention, the detailed description may be omitted, and the present invention is not limited thereby. Various modifications and applications of the present invention are possible within the scope of the claims described below and the equivalents interpreted therefrom.

In addition, the terms used herein are terms used to appropriately express preferred embodiments of the present invention, and may vary depending on the intention of the user or operator or the practices of the field to which the present invention pertains. Therefore, definitions of these terms should be made based on the content throughout the present invention. Throughout the present invention, when a part is said to “include” a certain component, unless specifically stated to the contrary, this means that it may further include other components rather than excluding other components.

All technical terms used in the present invention, unless otherwise defined, are used with the same meaning as commonly understood by one of ordinary skill in the art in the field related to the present invention. In addition, although preferred methods and samples are described in the present invention, similar or equivalent methods and samples are also included in the scope of the present invention. The contents of all publications incorporated in the present invention by reference are incorporated in the present invention.

The pathophysiology of sepsis is characterized by a systemic inflammatory response to infection. However, therapeutic attempts through cytokine blockade that targets a specific early inflammatory mediator, such as tumor necrosis factor, has failed. During sepsis, excessive endotoxins are internalized into the cytoplasm of immune cells, resulting in dysregulated pyroptotic cell death, which induces the leakage of late mediator alarmins such as HMGB1 and PTX3. As late mediators of lethal sepsis, overwhelming amounts of alarmins bind to high-affinity TLR4/MD2 and low-affinity RAGE receptors, thereby amplifying inflammation during early-stage sepsis.

In the present invention, a novel alarmin/receptor-targeting system using a TLR4/MD2/RAGE-blocking peptide (TMR peptide) obtained from a motif interacting with HMGB1/PTX3-receptors was developed. The TMR peptide successfully attenuates HMGB1/PTX3- and lipopolysaccharide (LPS)-regulated inflammatory cytokines by impairing their interactions with TLR4 and RAGE. Moreover, in the present invention, TMR peptide-conjugated liposomes (TMR-Lipo) were developed to improve peptide pharmacokinetics. In combination therapy, it was confirmed that moderately antibiotic-loaded TMR-Lipo exhibited a significant therapeutic effect in a mouse cecal ligation and puncture (CLP)-induced sepsis model.

The simultaneous release of multiple alarmins and subsequent activation of the alarmin/PRR axis during sepsis sustain strong host inflammatory responses, suggesting that immunomodulation by targeting the alarmin/PRR axis is a promising approach for sepsis therapy. In particular, a TMR peptide system derived from HMGB1, PTX3-TLR4/MD2, or RAGE interactions, specifically targeting TLR4/MD2 and RAGE, was identified. Through the present invention, it was confirmed that the TMR peptide significantly reduced TLR4- or RAGE-mediated secretion of inflammatory cytokines by specifically blocking HMGB1, PTX3-TLR4/MD2, or RAGE interactions.

In summary, the present invention shows that the TMR peptides according to the present invention have protective effects against sepsis, as evidenced by the results of the CLP-induced sepsis model.

Sepsis is a disorder characterized by immune dysregulation initiated by infection. Therefore, modulating hyperinflammatory responses is as important as effective infection control in bacterial sepsis. In CLP modeling studies according to one embodiment of the present invention, septic mice treated with antibiotics alone exhibited a slight survival benefit compared to septic mice treated with phosphate buffered saline (PBS) (FIGS. 5D to 5E), which is consistent with the findings showing the inadequacy of antibiotic treatment in treating sepsis. Interestingly, treatment with TMR-Lipo-Abs according to the present invention, which possess both antibacterial and immunomodulatory capacities, significantly reduced acute-phase mortality in septic mice in a dose-dependent manner (FIGS. 5D and 5E).

Clinical studies have shown that patients with sepsis have inconsistent and dysregulated immune systems, and thus precise immune modulation is important in sepsis treatment (R. Q. Yao, C. Ren, L. Y. Zheng, Z. F. Xia, Y. M. Yao, Advances in Immune Monitoring Approaches for Sepsis-Induced Immunosuppression, Front Immunol, 13 (2022) 891024.). Considering the role of the alarmin-PRR axis in sustaining inflammation during sepsis, targeting the alarmin-PRR signaling axis may provide a novel therapeutic method or therapeutic agent for sepsis. The TMR peptide according to the present invention is an antagonist of the alarmin-PRR axis that provides valuable insights into the modulation of hyperinflammation in sepsis and presents a potential therapeutic approach for sepsis treatment.

The release of HMGB1 into the blood contributes to lethality in both endotoxemia and sepsis. During lethal endotoxemia, HMGB1 binds to LPS, forming HMGB1-LPS complexes that are taken up by phagocytes through TLR4 and RAGE. Intracellular delivery of LPS activates caspase-11, a cytosolic endotoxin receptor, which subsequently induces pyroptosis. The caspase-11-mediated pyroptosis, a lytic form of cell death, disrupts the intracellular niche and rapidly releases cell contents, such as damage-associated molecular patterns (DAMPs), thereby increasing lethality in endotoxemia. Since extracellular LPS triggers pyroptosis of immune cells only after LPS has been delivered to caspase-11 inside the cell, the role of these alarmin proteins in delivering LPS into the cytosol through TLR4/RAGE represents a potential therapeutic target to prevent lethality in sepsis.

According to a previous study, HMGB1 has binding regions for the lipid A moiety of LPS at amino acids 80 to 96. In the present invention, it was confirmed through domain mapping that amino acids 89 to 98 of HMGB1 inhibit HMGB1-TLR4 interactions, and amino acids 160 to 169 of HMGB1 prevent HMGB1-RAGE interactions. These HMGB1-derived TLR4/RAGE targeting sequences may inhibit the delivery of LPS into the cytosol.

The TMR peptide according to the present invention is a fusion peptide in which four peptides consisting of amino acid sequences represented by SEQ ID NO: 2 to SEQ ID NO: 5 are connected by a linker. The linker may increase the flexibility of the fusion protein without interfering with the structure of each component in the fusion protein. In some embodiments, the linker residue is a peptide linker having a length of 2 to 100 amino acids. Exemplary linkers include linear peptides having at least two amino acid residues, such as Gly-Gly, Gly-Ala-Gly, Gly-Pro-Ala, Gly (G)ₙ, and Gly-Ser (GS) linkers. The GS linkers described herein include (GS)ₙ, (GSGSG)ₙ, (G₂S)ₙ, G₂S₂G, (G₂SG)ₙ, (G₃S)ₙ, (G₄S)ₙ, (GGSGG)ₙGₙ, GSG₄SG₄SG, and (GGGGS)ₙ, but are not limited thereto, and here n is 1 or greater. An example of the (G)ₙ linker includes a G₉ linker, and examples of the (GGGGS)ₙ linker include a GGGGS or (GGGGS)₃ linker. Suitable linear peptides include polyglycines, polyserines, polyprolines, polyalanines, and oligopeptides consisting of alanyl and/or serinyl and/or prolinyl and/or glycyl amino acid residues. The linker residue may be used to connect the components of the fusion proteins disclosed in the present invention. The present invention discloses a pharmaceutical composition for treating sepsis, including the above-described fusion protein. In one embodiment of the present invention, the fusion protein according to the present invention is a peptide consisting of an amino acid sequence represented by SEQ ID NO: 1.

The SEQ ID NOs indicated in the present invention are shown in Table 1.

TABLE 1

| SEQ | |
|---|---|
| ID NO. | Sequence |
| 1 | FKDPNAPKRPGGAAYRAKGKPDG |
| | GPAEARLTSALGGALAAVLEELR |
| 2 | FKDPNAPKRP |
| 3 | AAYRAKGKPD |
| 4 | PAEARLTSAL |
| 5 | ALAAVLEELR |

The term HMGB1 used in the present invention may be used interchangeably with SBP-1, HMG3, HMG1, high mobility group protein B1, high mobility group protein 1, DKFZp686A04236, amphoterin, HMG-1, high mobility group protein box 1, high mobility group protein (nonhistone chromosomal) protein 1, or sulfoglucuronyl carbohydrate binding protein, and in the present invention, a human-derived protein with accession number CAG33144 was used.

The term PTX3 used in the present invention may be used interchangeably with pentraxin 3, TSG-14, tumor necrosis factor-inducible gene 14 protein, TNFAIP5, tumor necrosis factor alpha-induced protein 5, pentraxin-related protein PTX3, TNF alpha-induced protein 5, long pentraxin 3, pentraxin-related gene, rapidly induced by IL-1 beta, pentaxin-related gene, rapidly induced by IL-1 beta, tumor necrosis factor, alpha-induced protein 5, tumor necrosis factor-inducible protein TSG-14, pentraxin-related protein PTX3, pentraxin 3, long, or TSG14, and in the present invention, a human-derived protein with accession number CA44778 was used.

The peptides H-T, H-R, P-T, and P-M consisting of amino acid sequences represented by SEQ ID NOs: 2 to 5 included in the TMR peptide of the present invention, may be arranged in the order of H-T, H-R, P-T, and P-M as in SEQ ID NO: 1, but the arrangement order is not limited thereto as long as each may implement the function of the peptide. For example, the TMR peptide according to the present invention may be arranged in the order of SEQ ID NO: 2, SEQ ID NO: 3, SEQ ID NO: 4, and SEQ ID NO: 5; SEQ ID NO: 2, SEQ ID NO: 3, SEQ ID NO: 5, and SEQ ID NO: 4; SEQ ID NO: 2, SEQ ID NO: 4, SEQ ID NO: 3, and SEQ ID NO: 5; SEQ ID NO: 2, SEQ ID NO: 4, SEQ ID NO: 5, and SEQ ID NO: 3; SEQ ID NO: 2, SEQ ID NO: 5, SEQ ID NO: 3, and SEQ ID NO: 4; SEQ ID NO: 2, SEQ ID NO: 5, SEQ ID NO: 4, and SEQ ID NO: 3; SEQ ID NO: 3, SEQ ID NO: 2, SEQ ID NO: 4, and SEQ ID NO: 5; SEQ ID NO: 3, SEQ ID NO: 2, SEQ ID NO: 5, and SEQ ID NO: 4; SEQ ID NO: 3, SEQ ID NO: 4, SEQ ID NO: 2, and SEQ ID NO: 5; SEQ ID NO: 3, SEQ ID NO: 4, SEQ ID NO: 5, and SEQ ID NO: 2; SEQ ID NO: 3, SEQ ID NO: 5, SEQ ID NO: 2, and SEQ ID NO: 4; SEQ ID NO: 3, SEQ ID NO: 5, SEQ ID NO: 4, and SEQ ID NO: 2; SEQ ID NO: 4, SEQ ID NO: 2, SEQ ID NO: 3, and SEQ ID NO: 5; SEQ ID NO: 4, SEQ ID NO: 2, SEQ ID NO: 5, and SEQ ID NO: 3; SEQ ID NO: 4, SEQ ID NO: 3, SEQ ID NO: 2, and SEQ ID NO: 5; SEQ ID NO: 4, SEQ ID NO: 3, SEQ ID NO: 5, and SEQ ID NO: 2; SEQ ID NO: 4, SEQ ID NO: 5, SEQ ID NO: 2, and SEQ ID NO: 3; SEQ ID NO: 4, SEQ ID NO: 5, SEQ ID NO: 3, and SEQ ID NO: 2; SEQ ID NO: 5, SEQ ID NO: 2, SEQ ID NO: 3, and SEQ ID NO: 4; SEQ ID NO: 5, SEQ ID NO: 2, SEQ ID NO: 4, and SEQ ID NO: 3; SEQ ID NO: 5, SEQ ID NO: 3, SEQ ID NO: 2, and SEQ ID NO: 4; SEQ ID NO: 5, SEQ ID NO: 3, SEQ ID NO: 4, and SEQ ID NO: 2; SEQ ID NO: 5, SEQ ID NO: 4, SEQ ID NO: 2, and SEQ ID NO: 3; or SEQ ID NO: 5, SEQ ID NO: 4, SEQ ID NO: 3, and SEQ ID NO: 2.

One embodiment of the present invention provides a nucleic acid molecule encoding a fusion protein or a recombinant vector including the nucleic acid molecule, and a recombinant strain including the nucleic acid molecule or the recombinant vector.

The fusion protein described herein may or may not include a signal peptide that functions to secrete the fusion protein from a host cell. A nucleic acid sequence encoding a signal peptide may be operably linked to a nucleic acid sequence encoding a protein of interest. In some embodiments, the fusion protein includes a signal peptide. In some embodiments, the fusion protein does not include a signal peptide.

In addition, the fusion protein described herein may include modified forms of protein binding peptides. For example, a fusion protein component may have post-translational modifications, including, for example, glycosylation, sialylation, acetylation, and phosphorylation on any of the protein binding peptides.

Unless otherwise specified, the fusion protein of the present invention is administered as a polypeptide (or a nucleic acid encoding a polypeptide) that is not in itself a part of a live, killed, or recombinant bacterial or viral vectored vaccine. In addition, unless otherwise specified, the fusion protein of the present invention is an isolated fusion protein, for example, not incorporated into the flagellum.

In the present invention, the term “fusion” refers to integrating two molecules with different or identical functions or structures, and may be fusion by any physical, chemical, or biological methods in which peptides may bind. The fusion protein or a polypeptide constituting the fusion protein may be prepared by a chemical peptide synthesis method known in the art, or by amplifying a gene encoding the fusion protein by polymerase chain reaction (PCR) or synthesizing it by a known method, and then cloning it into an expression vector and expressing it.

The term “polynucleotide” or “nucleic acid,” as used herein, is intended to include DNA and RNA, such as genomic DNA, cDNA, mRNA, recombinantly produced and chemically synthesized molecules. Nucleic acids may be single-stranded or double-stranded. RNA includes in vitro transcribed RNA (IVT RNA) or synthetic RNA. According to the present invention, the polynucleotide is preferably an isolated polynucleotide.

A nucleic acid may be included in a vector. The term “vector” as used herein includes any vector known to those skilled in the art, including a plasmid vector, a cosmid vector, a phage vector such as lambda phage, a viral vector such as a retrovirus, adenovirus or baculovirus vector, and an artificial chromosome vector such as a bacterial artificial chromosome (BAC), a yeast artificial chromosome (YAC) or a P1 artificial chromosome (PAC). The vector includes an expression vector as well as a cloning vector. An expression vector includes a viral vector as well as a plasmid vector, and generally includes an encoding sequence and a suitable DNA sequence required for expression of operably linked encoding sequences in a particular host organism (e.g., bacteria, yeast, plants, insects, or mammals) or in an in vitro expression system. Cloning vectors are generally used to engineer and amplify specific desired DNA fragments and may lack a functional sequence necessary for expressing the desired DNA fragments.

In one embodiment of all aspects of the present invention, a nucleic acid encoding the fusion protein according to the present invention is expressed in cells of an individual to be treated to produce the fusion protein according to the present invention. In one embodiment of all aspects of the present invention, a nucleic acid is transiently expressed in cells of an individual. Accordingly, in one embodiment, the nucleic acid is not inserted into the genome of the cells. In one embodiment of all aspects of the present invention, the nucleic acid is RNA, preferably RNA transcribed in vitro.

The nucleic acid described herein may be a recombinant and/or isolated molecule.

The term “RNA” as used herein relates to a nucleic acid molecule including ribonucleotide residues. In a preferred embodiment, an RNA encompasses all or most ribonucleotide residues. As used herein, “ribonucleotide” refers to a nucleotide having a hydroxy group at the 2′-position of the β-D-ribofuranosyl group. An RNA encompasses, but is not limited to, double-stranded RNA, single-stranded RNA, isolated RNA such as purified RNA, essentially pure RNA, synthetic RNA, recombinantly produced RNA, as well as naturally occurring RNA and modified RNA changed by addition, deletion, substitution and/or alteration of one or more nucleotides. These alterations may refer to the addition of non-nucleotide material to internal RNA nucleotides or to the RNA terminus (termini). In addition, it is considered that nucleotides in RNA may be chemically synthesized nucleotides or non-standard nucleotides such as deoxynucleotides. In the present invention, these altered RNAs are considered analogs of naturally occurring RNAs.

In specific embodiments of the present invention, the RNA is messenger RNA (mRNA), which is associated with an RNA transcript that encodes a peptide or protein. As established in the art, mRNA generally includes a 5′-untranslated region (5′-UTR), a peptide-coding region, and a 3′-untranslated region (3′-UTR). In some embodiments, the RNA is produced by in vitro transcription or chemical synthesis. In one embodiment, the mRNA is produced through in vitro transcription using a DNA template, and the DNA here refers to a nucleic acid containing deoxyribonucleotides.

In one embodiment, the RNA is in vitro transcribed RNA (IVT-RNA) and may be obtained by in vitro transcription from a suitable DNA template. The transcriptional regulatory promoter may be any promoter for any RNA polymerase. A DNA template for in vitro transcription may be obtained by cloning a nucleic acid, especially cDNA, and introducing it into an appropriate vector for in vitro transcription. cDNA may be obtained by reverse transcription of DNA.

In one embodiment, the RNA may have modified ribonucleotides. Examples of modified ribonucleotides include, but are not limited to, 5-methylcytidine, pseudouridine, and/or 1-methyl-pseudouridine.

In some embodiments, the RNA according to the present invention includes a 5′-cap. In one embodiment, the RNA herein does not have an uncapped 5′-triphosphate. In one embodiment, the RNA may be modified with a 5′-cap analog. The term “5′-cap” refers to a structure found at the 5′-end of mRNA, which generally consists of a guanosine nucleotide linked to the mRNA through a 5′->5′ triphosphate linkage. In one embodiment, this guanosine is methylated at the 7-position. RNA tagged with a 5′-cap or a 5′-cap analog may be provided by in vitro transcription in which the 5′-cap is co-transcriptionally expressed into an RNA strand, or by translating it using a capping enzyme and then attaching it to the RNA.

In some embodiments, the RNA according to the present disclosure includes a 5′-UTR and/or 3′-UTR. The term “untranslated region” or “UTR” refers to a region in a DNA molecule that is transcribed but not translated into an amino acid sequence, or a corresponding region in an RNA molecule, such as an mRNA molecule. A UTR may be present at 5′ (upstream) of an open reading frame (5′-UTR) and/or may be present at 3′ (downstream) of the open reading frame (3′-UTR). The 5′-UTR, when it is present, is located at the 5′ end, upstream of the start codon of a protein-encoding region. The 5′-UTR (when it is present) is present downstream of the 5′-cap, for example, immediately adjacent to the 5′-cap. The 3′-UTR, when it is present, is located at the 3′ end, downstream of the stop codon of a protein-encoding region, but the “3′-UTR” preferably does not include a poly(A) tail. Therefore, the 3′-UTR (when it is present) is present upstream of the poly(A) sequence, e.g., immediately adjacent to the poly(A) sequence.

In some embodiments, the RNA according to the present invention includes a 3′-poly(A) sequence. As used herein, the term “poly(A) sequence” or “poly-A tail” refers to a sequence of uninterrupted or interrupted adenyl (A) residues typically located at the 3′ end of an RNA molecule. The poly(A) sequence follows the 3′-UTR in the RNAs that are known to those skilled in the art and described herein. The poly(A) sequence may be of any length. In some embodiments, the poly(A) sequence includes or consists of at least 20, at least 30, at least 40, at least 80, or at least 100, and at most 500, at most 400, at most 300, at most 200, or at most 150 A nucleotides, and, in particular, about 110 A nucleotides.

In some embodiments, the poly(A) sequence consists only of A nucleotides. In some embodiments, as disclosed in WO 2016/005324 A1, incorporated herein by reference, the poly(A) sequence consists essentially of A nucleotides, but is interrupted by a random sequence of four types of nucleotides (A, C, G, and U). This random sequence may be 5 to 50, 10 to 30, or 10 to 20 nucleotides long. A poly(A) cassette that is present in an encoding sequence of a DNA essentially consisting of dA nucleotides but interrupted by a random sequence having length of, for example, 5 to 50 nucleotides with equivalent distribution of the four types of nucleotides (dA, dC, dG, dT) exhibits, at the DNA level, steady propagation of plasmid DNA in E. coli, and at the RNA level, it is still associated with beneficial properties in terms of supporting RNA stability and translation efficiency.

In some embodiments, no nucleotide other than an A nucleotide flanks the poly(A) sequence at its 3′-end, that is, no nucleotide other than A masks or follows the poly(A) sequence at its 3′-end.

In the context of the present invention, the term “transcription” refers to the process in which a genetic code is transcribed from a DNA sequence into RNA. Thereafter, the RNA may be translated into a peptide or protein.

“Encoding” refers to the inherent property of a specific sequence of nucleotides in a polynucleotide, such as a gene, cDNA, or mRNA, that has a defined sequence of nucleotides (i.e., rRNA, tRNA, and mRNA) or a defined sequence of amino acids and biological properties derived therefrom and that allows the sequence to serve as a template for synthesis of other polymers and macromolecules in biological processes. Therefore, when transcription and translation of mRNA corresponding to the gene produce a protein in a cell or other biological system, the gene encodes a protein. Both a coding strand, which is identical to an mRNA sequence and is generally provided in a sequence listing, and a non-coding strand, which is used as a template for transcription of a gene or cDNA, may be mentioned as encoding a protein or another product of the gene or cDNA.

As used herein, the term “endogenous” refers to any substance derived or produced within an organism, cell, tissue, or system.

As used herein, the term “exogenous” refers to any substance derived or produced outside an organism, cell, tissue, or system.

As used herein, the term “expression” is defined as transcription and/or translation of a specific nucleotide sequence.

As used herein, the terms “linked” and “fused” or “fusion” are used interchangeably. These terms mean two or more elements or components or domains linked together.

The peptides, proteins, polypeptides, RNA, RNA particles, and additional substances described herein may be administered as a pharmaceutical composition or medicament for therapeutic or prophylactic treatment, and may be administered in the form of any appropriate pharmaceutical composition that may include a pharmaceutically acceptable carrier or that may optionally include one or more of adjuvants, stabilizers, and the like. In one embodiment, the pharmaceutical composition is for use in therapeutic or prophylactic treatment, for example, in treating or preventing an antigen-associated disease, such as cancer, such as those described herein.

The term “pharmaceutical composition” relates to a formulation including a therapeutically effective substance, preferably together with pharmaceutically acceptable carriers, diluents, and/or excipients. The pharmaceutical composition is useful for reducing the severity of, preventing, or treating a disease or disorder by administering the pharmaceutical composition to an individual. The pharmaceutical composition is also known in the art as a pharmaceutical formulation. In the context of the present invention, the pharmaceutical composition includes peptides, proteins, polypeptides, RNA, RNA particles, immune effector cells, and/or additional substances as described herein.

The pharmaceutical composition of the present invention may include one or more adjuvants or may be administered together with one or more adjuvants. The term “adjuvant” refers to a compound that prolongs, enhances, or accelerates an immune response. Adjuvants include a heterogeneous group of compounds such as oil emulsions (e.g., Freund's adjuvant), mineral compounds (e.g., alum), bacterial products (e.g., pertussis toxin) or immune stimulating complexes. Examples of adjuvants include LPS, glycoprotein 96 (GP96), cytosine-phosphate-guanine (CpG) oligodeoxynucleotides, growth factors, and cytokines such as monokines, lymphokines, interleukins, and chemokines, but are not limited thereto. The cytokine may be IL1, IL2, IL3, IL4, IL5, IL6, IL7, IL8, IL9, IL10, IL12, IFNα, IFNγ, granulocyte-macrophage colony-stimulating factor (GM-CSF), and lymphotoxin-alpha (LT-a). Additional known adjuvants are aluminum hydroxide, Freund's adjuvant, or oils such as Montanide® ISA51.

The pharmaceutical composition according to the present invention is generally applied in a “pharmaceutically effective amount” and as a “pharmaceutically acceptable formulation.”

The term “pharmaceutically acceptable” refers to the non-toxic nature of a substance that does not interact with the action of an active ingredient of a pharmaceutical composition.

The term “pharmaceutically effective amount” or “therapeutically effective amount” refers to the amount that, alone or in combination with additional administration, achieves the desired response or desired effect. When treating a particular disease, the desired response preferably refers to inhibition of progression of the disease. This includes slowing down the progression of the disease, and in particular, discontinuing or reversing the progression of the disease. The desired response in the treatment of a disease may also be delaying the onset or preventing the onset of the disease or condition. The effective amount of the composition described herein will be determined depending on the condition being treated, the severity of the disease, the individual characteristics of the patient, including age, physical condition, height, and weight, the duration of treatment, the type of concomitant therapy (if any), the specific route of administration, and similar factors. Therefore, the administered dosage of the compositions described herein may be determined according to these various properties. When the response in the patient is not sufficient with the first dose, higher doses (or effectively, higher doses achieved by other, more local routes of administration) may be used.

The pharmaceutical composition of the present invention may include salts, buffers, preservatives, and optionally other therapeutic agents. In one embodiment, the pharmaceutical composition of the present invention include one or more pharmaceutically acceptable carriers, diluents, and/or excipients.

Preservatives suitable for use in the pharmaceutical composition of the present invention include benzalkonium chloride, chlorobutanol, parabens, and thimerosal, but are not limited thereto.

The term “excipient” as used herein refers to a substance that may be present in the pharmaceutical composition of the present invention but is not an active ingredient. Examples of excipients include carriers, binders, diluents, lubricants, thickeners, surfactants, preservatives, stabilizers, emulsifiers, buffers, flavoring agents, or colorants, but are not limited thereto.

The term “diluent” refers to a substance that dilutes and/or thins. In addition, the term “diluent” includes any one or more of fluids, liquids or solid suspensions, and/or mixed media. Examples of suitable diluents include ethanol, glycerol, and water.

The term “carrier” refers to an ingredient, which may be natural, synthetic, organic, or inorganic, that is combined with an active ingredient to facilitate, enhance, or allow the administration of the pharmaceutical composition. As used herein, a carrier may be one or more compatible solid or liquid fillers, diluents, or encapsulating materials suitable for administration to an individual. Suitable carriers include sterile water, Ringer's solution, Ringer's lactate, sterile sodium chloride solution, isotonic saline, polyalkylene glycols, hydrogenated naphthalenes, and, especially, biocompatible lactide polymers, lactide/glycolide copolymers, or polyoxyethylene/polyoxypropylene copolymers, but are not limited thereto. In one embodiment, the pharmaceutical composition of the present invention includes isotonic saline. In one embodiment of the present invention, the TMR peptide may be delivered by encapsulation, and the encapsulation is implemented through liposomes.

Pharmaceutically acceptable carriers, excipients, or diluents for therapeutic use are well known in the pharmaceutical field and are described, for example, in Remington's Pharmaceutical Sciences, Mack Publishing Co. (A. R Gennaro edit. 1985).

Pharmaceutical carriers, excipients, or diluents may be selected depending on the intended route of administration and standard pharmaceutical practices.

In one embodiment, the pharmaceutical composition described herein may be administered intravenously, intraarterially, subcutaneously, intradermally, or intramuscularly. In specific embodiments, the pharmaceutical composition is formulated for topical or systemic administration. Systemic administration may include enteral administration involving absorption through the gastrointestinal tract or parenteral administration. As used herein, the term “parenteral administration” refers to administration in any manner other than through the gastrointestinal tract, such as by intravenous injection. In a preferred embodiment, the pharmaceutical composition is formulated for systemic administration. In another preferred embodiment, the systemic administration is by intravenous administration. In one embodiment of all aspects of the invention, the RNA encoding the TMR fusion protein described herein and optionally the RNA encoding an antigen are administered systemically.

The term “co-administration” as used herein refers to the administration of multiple compounds or compositions (e.g., immune effector cells, RNA encoding an IL2 variant polypeptide, and optionally RNA encoding a vaccine antigen) to the same patient. The multiple compounds or compositions may be administered simultaneously, essentially simultaneously, or sequentially.

The materials, compositions, and methods described herein may be used to treat individuals with a disease, for example, a disease characterized by the presence of diseased cells that express inflammation. A particularly preferred diseases is sepsis.

The materials, compositions, and methods described herein may be used in the therapeutic or prophylactic treatment of a variety of diseases, and as described herein, the support of and/or the activation of immune effector cells is advantageous for patients with inflammatory diseases or cancer.

The term “disease” refers to an abnormal condition affecting an individual's body. A disease is often interpreted as a medical condition associated with specific symptoms and signs. A disease may be caused by an agent originating from an external source such as an infectious disease, or may be caused by an internal dysfunction such as an autoimmune disease. In humans, “disease” is used in a broader sense to refer to any condition that, upon contact with an individual, causes pain, dysfunction, distress, social problems, death, or similar problems in the afflicted individual. In a broader sense, it sometimes includes injuries, disabilities, impairments, syndromes, infections, isolated symptoms, deviant behavior, and atypical variants, both structural and peptide combinations, and in other contexts and for other purposes, it is considered a distinct category. A disease generally affects an individual not only physically but also emotionally, as living with various diseases may change an individual's perspective on life and personality.

In this context, the terms “treatment,” “treating,” or “therapeutic intervention” refer to the management and care of an individual for the purpose of combating a condition such as a disease or disorder. These terms are intended to include not only ameliorating symptoms or complications, delaying the progression of a disease, disorder, or condition, ameliorating or alleviating symptoms and complications, and/or curing or eliminating a disease, disorder, or condition, but also the full-range treatment for a given condition from which an individual suffers, such as the administration of a therapeutically effective compound to prevent a condition, and here, prevention will be understood as the management and care of an individual for the purpose of combating a disease, condition, or disorder, and includes the administration of an active compound to prevent the onset of symptoms or complications.

The term “therapeutic treatment” refers to any treatment that improves the health status of an individual and/or prolongs (increases) the lifespan of the individual. The treatment may be eliminating a disease in an individual, stopping or slowing down the progression of a disease in an individual, inhibiting or slowing down the progression of a disease in an individual, reducing the frequency or severity of symptoms in an individual, and/or reducing the relapse of a disease in an individual who is currently suffering from the disease or has previously suffered from the disease.

The term “prophylactic treatment” or “preventive treatment” refers to any treatment intended to prevent a disease from developing in an individual. The term “prophylactic treatment” or “preventive treatment” are used interchangeably herein.

Improvement may be indicated by an improvement in the disease activity index, by amelioration of clinical symptoms, or by any other measure of disease activity. One such disease index is the Mayo Score for Ulcerative Colitis. The Mayo Score is an established, validated disease activity index for mild, moderate, and severe ulcerative colitis (UC) calculated as the sum of four sub-scores of stool frequency, rectal bleeding, endoscopy findings, and Physician's Global Assessment (PGA), and it ranges from 0 to 12. A score of 3 to 5 points indicates mildly active disease, a score of 6 to 10 points indicates moderately active disease, and a score of 11 to 12 points indicates severe disease. The partial Mayo score, which is the Mayo score without the endoscopy sub-score, is calculated as the sum of the stool frequency, rectal bleeding, and PGA sub-scores and ranges from 0 to 9. The modified Mayo score, which is the Mayo score without the PGA sub-score, is calculated as the sum of the stool frequency, rectal bleeding, and endoscopy sub-scores and ranges from 0 to 9. Other disease activity indices for UC include, for example, the Ulcerative Colitis Endoscopic Index of Severity (UCEIS) score and the Bristol Stool Form Scale (BSFS) score. The UCEIS score provides an overall assessment of the endoscopic severity of UC based on mucosal vascular patterns, bleeding, and ulceration (Travis et al., Gut. 61:535-542 (2012)). The score ranges from 3 to 11, and a higher score indicates more severe disease based on endoscopy. The BSFS score is used to classify the form (or morphology) of human excrement into seven categories (Lewis and Heaton, Scand J Gastroenterol. 32(9): 920-924 (1997)).

The terms “subject” and “individual” are used interchangeably herein. These terms refer to humans or other mammals (e.g., mice, rats, rabbits, dogs, cats, cows, pigs, sheep, horses, or primates) that may suffer from or may be susceptible to a disease or disorder (e.g., cancer), but they may or may not suffer from the disease or disorder. In many embodiments, the individual is a human. Unless otherwise specified, the terms “subject” and “individual” do not refer to a specific age and therefore encompass adults, the elderly, children, and newborns. In embodiments of the present invention, an “individual” or “subject” is a “patient.”

The term “patient” refers to a subject or individual in need of treatment, specifically a subject or individual suffering from a disease.

In one embodiment of the present invention, the goal is to treat a disease by inhibiting the expression of inflammatory cytokines in inflammatory cells expressing inflammation, increasing the survival rate of individuals with inflammatory cells, killing cancer cells, or inhibiting the proliferation of cancer cells, or increasing the survival rate of individuals bearing cancer cells.

### EXAMPLES

### Example 1. Materials and Methods

### 1.1 Mice and Cell Culture

Primary bone marrow-derived macrophages (BMDMs) were isolated from female C57BL/6 mice and cultured for five to seven days in a medium supplemented with macrophage-colony stimulating factor (M-CSF) for differentiation. HEK293T cells were acquired from the American Type Culture Collection (ATCC). Both BMDMs and HEK293T cells were cultured in Dulbecco's modified Eagle's medium (DMEM, GenDEPOT Inc., USA) supplemented with 10% fetal bovine serum (FBS), penicillin (100 U/mL), and streptomycin (100 μg/mL).

Wild-type female C57BL/6 mice were obtained from Samtako Bio Korea Inc. (Osan, Republic of Korea) and raised under standard conditions at the Center for Laboratory Animal Science at Hanyang University (Ansan, Republic of Korea). All animal breeding and experimental procedures were conducted according to the guidelines approved by the Institutional Animal Care and Use Committee (IACUC) of Hanyang University (protocol 2022-0004).

### Example 1.2. Plasmid Design

Flag-HMGB1 (MG50913-CF), Flag-PTX3 (HF12082-CF), HA-TLR4 (MG50657-NY), Myc-RAGE (MG50489-CM), and His-MD2 (MG51098-NH) plasmids were purchased from Sino Biological Inc. (Beijing, China). Plasmids encoding different regions of HMGB1 or PTX3 were produced by PCR amplification of full-length HMGB1 or PTX3 cDNA and subcloned into a pEBG derivative encoding an N-terminal glutathione s-transferase (GST)-epitope tag located between the BamHI and NotI sites. All constructs were sequenced to confirm 100% matching with the original sequence.

### Example 1.3. Recombinant Protein

To acquire recombinant rFlag-HMGB1, rFlag-PTX3, rHA-TLR4, rMyc-RAGE, and rHis-MD2 were cloned with an N-terminal tag into a pRSFDuet-1 vector (Novagen Inc., USA). Thereafter, BL21 (DE3) pLysS was expressed, harvested, and purified according to the standard procedures recommended by Novagen Inc. The purified rFlag-HMGB1, rFlag-PTX3, rHA-TLR4, rMyc-RAGE, and rHis-MD2 were analyzed with a permeable cellulose membrane, examined for lipopolysaccharide contamination using a limulus amebocyte lysate (LAL) assay (BioWhittaker Inc.), which showed that less than 20 μg/mL of lipopolysaccharide was included.

### Example 1.4. Peptides

HMGB1 and PTX3 peptides were industrially synthesized and purified in the form of an acetate salt form by Lugen Science Inc. (Republic of Korea) to prevent abnormal reactions in cells. The peptide sequences used in the present invention are shown in FIGS. 2A, 2B, and 3A.

### Example 1.5. GST Pulldown and Immunoprecipitation Analyses

HEK293T cells and BMDMs were treated as specified and analyzed using GST pulldown and co-immunoprecipitation assays. For GST pulldown, HEK293T cells were harvested and lysed in radio immunoprecipitation assay (RIPA) buffer containing a complete protease inhibitor cocktail (GenDEPOT Inc.). After centrifugation, the supernatant was mixed with a 50% suspension of glutathione-conjugated Sepharose beads (Amersham Biosciences, Amersham, UK), and the resulting mixture was incubated for one hour at 4° C. The precipitate was washed repeatedly with RIPA buffer. Proteins bound to the glutathione beads were eluted with a sodium dodecyl sulfate (SDS) buffer by boiling for five minutes.

For immunoprecipitation, HEK293T cells and BMDMs were harvested and lysed in RIPA buffer containing a complete protease inhibitor cocktail (GenDEPOT Inc., USA). After pre-washing protein A/G agarose beads for one hour at 4° C., lysates were used for immunoprecipitation with the specified antibodies. 1 mL of cell lysates were treated with 1 to 4 μg of a commercially available antibody, and the resulting product was incubated at 4° C. overnight. After treating with protein A/G agarose beads for two hours, the immunoprecipitants were repeatedly washed with RIPA buffer and eluted with SDS buffer by boiling for five minutes.

### Example 1.6. Immunoblotting

For immunoblotting, the proteins were resolved by sodium dodecyl sulfate-polyacrylamide gel electrophoresis (SDS-PAGE) and transferred onto a polyvinylidene difluoride (PVDF) membrane (IPVH00010, Merck Inc., USA). Anti-HMGB1 (ab228624, Abcam Inc., UK), anti-PTX3 (sc-373951, SANTA CRUZ BIOTECHNOLOGY Inc., USA), anti-Flag (2368S, Cell Signaling Technology (CST) Inc., USA), anti-HA (3724S, CST Inc., USA), anti-Myc (2278S, CST Inc., USA), anti-His (12698S, CST Inc., USA), anti-GST (2625S, CST Inc., USA), anti-TLR4 (ab95562, Abcam, UK), anti-RAGE (sc-365154, SANTA CRUZ BIOTECHNOLOGY Inc., USA), anti-MD2 (MA5-15766, Invitrogen, USA), anti-MyD88 (sc-74532, SANTA CRUZ BIOTECHNOLOGY Inc., USA), anti-TRAF6 (sc-8409, SANTA CRUZ BIOTECHNOLOGY Inc., USA), anti-IRAK (sc-5288, SANTA CRUZ BIOTECHNOLOGY Inc., USA), anti-NF-κ (sc-372, SANTA CRUZ BIOTECHNOLOGY Inc., USA), anti-KRAS (ab206969, Abcam, UK), anti-p-p38 MAPK (MA5-15182, Invitrogen, USA), anti-β-Actin (sc-47778, SANTA CRUZ BIOTECHNOLOGY Inc., USA), and anti-GAPDH (sc-32233, SANTA CRUZ BIOTECHNOLOGY Inc., USA) were incubated at 4° C. for more than 16 h.

Antibody conjugation was visualized using EzWestLumi Plus (ATTO Inc., Japan) and detected using a Vilber chemiluminescence analyzer (Fusion Solo; Vilber Lourmat SAS, France).

### Example 1.7. Enzyme-Linked Immunosorbent Assay

The cytokine content in the supernatants of the cultured BMDMs and the serum of CLP-induced mice was measured using the uncoated ELISA Kit for the detection of TNF-α (88-7324-88, Invitrogen, USA), IL-6 (88-7064-88, Invitrogen), IL-10 (88-7105-88, Invitrogen). The IL-12p40 cytokine was measured using the OptEIA™ Mouse IL-12 p40 ELISA Set (555165, BD Biosciences, USA). All experiments were conducted following the guidelines provided by the manufacturer.

### Example 1.8. In Vitro Cell Viability Study

BMDMs were plated on 96-well plates (SPL Life Science Co. Ltd., Republic of Korea) at a density of 2×10⁴ cells/well and incubated in complete DMEM for 18 h. Thereafter, the cells were treated with various concentrations of H-T, H-R, P-T, P-M peptides, control, TMR, or TMR using Abs Lipo. After 48 h of incubation, the medium was replaced with 100 μl of DMEM and the Quanti-Max solution (10% of each media volume). After two hours of incubation at 37° C. and 5% CO₂, the absorbance of the plates including the Quanti-Max solution was measured at 450 nm using the MMR SPARK® microplate reader (TECAN Group Ltd., Switzerland).

### Example 1.9. Preparation and Characterization of Liposomes

The present inventors prepared 0.5% by weight of liposomes using the TMR peptide, according to a previous study (E. Cho, S. J. Mun, M. Jeon, H. K. Kim, H. Baek, Y. S. Ham, W. J. Gil, J. W. Kim, C. S. Yang, Tumor-targeted liposomes with platycodin D2 promote apoptosis in colorectal cancer, Mater Today Bio, 22 (2023) 100745). Dipalmitoylphosphatidyl-choline (DPPC) (850355C, Avanti Polar Lipids Inc., USA) and poly(ethylene glycol) (PEG) (81188; Sigma-Aldrich) were dissolved in chloroform in a round-bottomed flask. The mixing ratio of DPPC and PEG was adjusted to 9:1, and the concentration of the TMR peptide was set to 1 mM. For peptide conjugation, PEG and DSPE-PEG2000-maleimide (880126C, Avanti Polar Lipids Inc., USA) were mixed at a ratio of 75:25. Thereafter, the solvent was evaporated in a rotary evaporator at 42° C. for 90 min to produce a thin film at the bottom of the flask. PBS was then added to the round-bottomed flask, and the film was hydrated in a water sonicator at 42° C. for 90 min. To conjugate the TMR peptide with the malemide co-assembled with the vesicular membrane, TMR-cysteamide was added to the liposome, and the resulting mixture was rotated at 4° C. overnight. The hydrodynamic particle sizes of control liposome (C-Lipo), TMR liposome (TMR-Lipo) and antibiotic-loaded TMR liposome (TMR-Lipo-Abs) were determined by dynamic light scattering (DLS 1070, Malvern, United Kingdom) at 25° C. The morphologies of C-Lipo, TMR-Lipo, and TMR-Lipo-Abs were observed using a transmission electron microscope (JEM 1010, JEOL CO. LTD., Japan).

To determine the encapsulation rate of TMR-Lipo, fluorescein isothiocyanate (FITC) was equimolarly conjugated to the TMR peptide (TMR peptide-FITC) using the FluoroTag™ FITC Conjugation Kit (FITC1, Merck, USA). Using the above-described method, liposomes containing TMR peptide-FITC conjugates were produced at various concentrations. The prepared liposomes were centrifuged at 27,000×g for two hours to separate the liposome pellets from the supernatant, and the level of residual TMR peptide-FITC in each supernatant was measured. The fluorescence intensity of the TMR peptide-FITC standard was measured using a microplate reader in the range of 485 nm to 535 nm. Finally, the conjugation efficiency was calculated as ‘Conjugation efficiency (%)=(1−(TMR peptide-FITC in supernatant/total TMR peptide-FITC))×100.’

### Example 1.10. Construction of Mouse Sepsis Model

Cecal ligation and puncture (CLP) was performed on six-week-old C57BL/6 female mice according to previously established protocols (D. Lee, E. Lee, S. Jang, K. Kim, E. Cho, S. J. Mun, W. Son, H. I. Jeon, H. K. Kim, Y. J. Jeong, Y. Lee, J. E. Oh, H. H. Yoo, Y. Lee, S. J. Min, C. S. Yang, Discovery of Mycobacterium tuberculosis Rv3364c-Derived Small Molecules as Potential Therapeutic Agents to Target SNX9 for Sepsis, J Med Chem, 65 (2022) 386-408.). To perform CLP, mice were anesthetized with 2,2,2-tribromoethanol (250 mg/kg, i.p.), and a small midline incision was made in the abdominal region to expose the cecum. The cecum was ligated below the ileocecal valve, punctured twice through both surfaces using a 22-gauge needle, and the abdominal incision was sutured. Survival rate was monitored daily for 3 and 10 days.

### Example 1.11. Histological Analysis

For the immunohistochemical analysis of mouse tissues, the lungs, liver, and spleen were fixed in 10% formalin and then embedded in paraffin. Paraffin sections (4 μm) were prepared and subjected to hematoxylin and eosin (H&E) staining. The histopathological score was determined based on the distribution and severity of inflammation within the tissues and cells. A board-certified pathologist independently confirmed each tissue section without prior knowledge of the group.

### Example 1.12. Statistical Analysis

All data is presented as mean±SD or SEM and was assessed using the Student's t-test with a Bonferroni adjustment or ANOVA for multiple comparisons. Statistical analyses were performed using GraphPad Prism software (version 8.0; La Jolla, CA, USA). Differences were considered statistically significant at p<0.05. For comparisons using GraphPad Prism, survival data was analyzed and visualized using the Kaplan-Meier product limit method with the log-rank (Mantel-Cox) test.

### Example 2. Results

### Example 2.1. Inflammation Ameliorating Effect of TLR4/RAGE-Mediated Signaling Through its Interaction with Receptors of Alarmins HMGB1 and PTX3

HMGB1 and PTX3 are ubiquitously expressed alarmins released from immune cells during the septic response. Macrophages are key innate immune players and are a major source of cytokines when they are activated by LPS. BMDMs were stimulated with LPS in a time-dependent manner, and the amount of HMGB1 and PTX3 secretion was analyzed by immunoblotting. In the LPS-stimulated BMDMs, the intracellular expression of HMGB1 and PTX3 increased after approximately two to four hours, and distinctive secretion occurred approximately 12 h later (FIGS. 1A and 1B).

Since the alarmin-PRR axis is critically involved in sepsis pathology, it was further investigated through co-immunoprecipitation whether HMGB1 and PTX3 physically interact with TLR4 or RAGE and whether this interaction contributes to inflammatory responses. In HEK293T cells, overexpressed Flag-HMGB1 interacted with both HA-TLR4 and Myc-RAGE (FIG. 1C, left panel). In addition, recombinant Flag-HMGB1 interacted with both intracellular TLR4 and RAGE in the BMDMs (FIG. 1C, right panel). Regarding PTX3, previous studies have reported its interaction with the TLR4/MD2 complex but not with RAGE. Verification of this interaction by co-immunoprecipitation showed that ectopically expressed Flag-PTX3 interacted with the TLR4/MD2 complex in HEK293T cells (FIG. 1D, left panel), and recombinant Flag-PTX3 also interacted with intracellular TLR4/MD2 in BMDMs (FIG. 1D, right panel), which was consistent with previous studies.

To determine whether these physical interactions between alarmins and PRRs are functionally related to their roles in inflammatory responses, the levels of inflammatory cytokines induced by the activation of TLR4 and RAGE signaling were assessed by stimulating murine BMDMs with LPS or recombinant alarmin proteins (rHMGB1 or rPTX3). rHMGB1 or rPTX3-treated BMDMs exhibited elevated levels of inflammatory cytokines, including TNF-α, IL-6, and IL-12p40, as well as elevated levels of anti-inflammatory cytokine IL-10, but the expression levels were slightly lower than that of LPS alone (FIG. 1E). In particular, combining LPS with rHMGB1 or rPTX3 significantly increased the production of these cytokines compared with LPS alone. These findings suggest that alarmin proteins contribute to the amplification of sterile inflammation. In summary, extracellular HMGB1 or PTX3 amplified inflammatory responses by interacting with their cognate receptors TLR4 and RAGE in the LPS-treated BMDMs.

### Example 2.2. Inhibitory Effects of HMGB1/PTX3-Derived Peptides on TLR4/RAGE Signaling

The data disclosed herein suggest that HMGB1 interacts with TLR4 and RAGE, and that PTX3 interacts with TLR4/MD2, thereby amplifying inflammatory responses under LPS treatment. According to a previous study, HMGB1 has two distinct binding regions within its domain: one for TLR4 binding (residues 89 to 108) and the other for RAGE binding (150 to 183) (H. E. Harris, U. Andersson, D. S. Pisetsky, HMGB1: a multifunctional alarmin driving autoimmune and inflammatory disease, Nat Rev Rheumatol, 8 (2012), 195-202.). To further identify the amino regions of HMGB1 essential for TLR4 or RAGE interactions, GST pulldown analyses were performed on HEK293T cell lysates expressing GST-tagged HMGB1 and its binding regions.

As a result, it was found that amino acids 89 to 98 of HMGB1 were essential for TLR4 binding, while amino acids 160 to 169 were essential for RAGE binding (FIG. 2A). Myeloid differentiation protein 2 (MD2), which is a co-receptor for TLR4, is essential for the PTX3-TLR4 interaction and subsequent TLR4 activation. Therefore, for mapping TLR4 and MD2 binding sites within PTX3, truncated variants of PTX3 (each truncate consisting of 10 aa portions from the aa 18 to 182 region of PTX3) were expressed in HEK293 cells, and GST pulldown was performed from the cell lysates. As a result, it was confirmed that aa 108 to 117 of PTX3 interacted with TLR4, and aa 148 to 157 interacted with MD2 (FIG. 2B).

Based on the potential TLR4-, MD2-, and RAGE-interacting motifs identified in each alarmin, the present inventors hypothesized that these motifs could compete with their parent motifs for PRR binding, thereby reducing downstream inflammatory responses. To test this hypothesis, antagonistic peptides against each PRR were synthesized based on the sequence of TLR4-, MD2-, and RAGE-interacting motifs (designated H-T, H-R, P-T, and P-M peptides). The synthesized peptides had almost no impact on the viability of BMDMs (FIG. 2C). These peptides successfully blocked alarmins-PRR interactions, as found by a cell-free immunoprecipitation assay (FIG. 2D). In addition, these peptides inhibited the binding of recombinant HMGB1 or PTX3 to their cognate receptors, which are expressed in macrophages (FIG. 2E). Simultaneously, the antagonistic effect of these peptides on blocking alarmin-PRR interactions resulted in a reduction in the production of inflammatory cytokines, without changes in IL-10 levels (FIG. 2F). These findings indicate that these peptides serve as antagonistic peptides against TLR4- and RAGE-mediated signaling.

### Example 2.3. Inhibitory Effects of TMR-Lipo on Alarmin-PRR Signaling Pathway

In sepsis, alarmin-mediated activation of PRRs, especially TLR4 and RAGE, stimulates inflammatory cytokine production by macrophages and other immune cells, resulting in excessive tissue inflammation. The present inventors hypothesized that targeting the dual alarmin-PRR axis (HMGB-1-TLR4/RAGE or PTX3-TLR4/MD2) using TLR4/MD2- and RAGE-interacting motifs could regulate exacerbated inflammatory responses in sepsis.

To test the hypothesis, a TLR4/MD2-RAGE multiblocking TMR peptide was designed by covalently conjugating TLR4/MD2 and RAGE-interacting motifs. Based on this peptide, a TMR peptide-tagged liposome (TMR-Lipo) was developed to overcome unfavorable pharmacokinetic properties (FIG. 3A). To achieve high encapsulation efficiency and uniform particle size distribution, liposomes were prepared using a thin-film hydration method. The characteristic vesicular structure of C-Lipo and TMR-Lipo was confirmed through TEM (FIG. 3B). C-Lipo showed an average hydrodynamic particle size of 142.02 nm, and a polydispersity index (PDI) of approximately 0.247. TMR-Lipo showed a hydrodynamic particle size of 141.74 nm and a PDI value of approximately 0.248 (FIG. 3C), which were similar to those of C-Lipo. In DSPE-PEG2000-maleimide, the hydrophilic maleimide head group was exposed to the aqueous phase, where the thiol-terminated TMR peptide was bound through a thiol-maleimide reaction to produce TMR-Lipo.

To determine the conjugation efficiency of the TMR peptide on the surface of liposomes, the encapsulation efficiency of the TMR peptide in TMR-Lipo was normalized by conjugating FITC to the TMR peptide, by referring to a previous study. In TMR-Lipo, the encapsulation efficiency of the TMR peptide-FITC began to slightly decrease at the concentration of 20 mM of the conjugated TMR peptide. When conjugated in mass units, the encapsulation efficiency decreased at 1 mg. The appropriate concentration of the TMR peptide for conjugation in the preparation of TMR-Lipo was 20 mM (FIG. 3D).

Next, the viability of macrophages treated with C-Lipo or TMR-Lipo was measured. As a result of evaluating the TMR-Lipo viability in a dose-dependent manner, its high biostability was confirmed in macrophages, similar to that of C-Lipo (FIG. 3E). After confirming that HMGB1 and PTX3 are released in LPS-treated macrophages and that alarmin-PRR interactions are increased (FIGS. 1A to 1D), it was investigated whether TMR-Lipo could block the intracellular binding of HMGB1 or PTX3 with TLR4 or RAGE in LPS-treated macrophages. To test this, LPS-primed BMDMs were exposed to TMR-Lipo, and then co-immunoprecipitation was performed with TLR4 or RAGE. As shown in FIG. 4A, the intracellular binding of secreted HMGB1 or PTX3 to their receptors was inhibited in the LPS-primed BMDMs upon TMR-Lipo treatment. Inflammatory activation of macrophages by LPS triggered NF-κB activation through TLR4/RAGE signaling and then induced the production of inflammatory cytokines (FIG. 4B).

To investigate whether TMR-Lipo regulates TLR4 or RAGE-mediated NF-κB activation and subsequent inflammatory responses, the expression levels of TLR4/RAGE-related downstream signaling molecules were detected by WB analysis in BMDMs treated with TMR-Lipo. It was found that the TMR-Lipo reduced the levels of TLR4- and RAGE-mediated downstream signaling molecules in a dose-dependent manner and decreased the intracellular levels of HMGB1 and PTX3 (FIG. 4C). In addition, TMR-Lipo significantly reduced inflammatory cytokines, including TNF-α, IL-6, and IL-12, while increasing anti-inflammatory IL-10 (FIG. 4D). In summary, the data showed that TMR-Lipo modulates LPS-induced inflammatory responses in macrophages.

### Example 2.4. In Vivo Analysis of the Therapeutic Effect of TMR-Lipo on Inflammation and Sepsis

Controlling both infection and inflammation are the two major goals of sepsis therapy. To accomplish these two goals simultaneously, the broad-spectrum antibiotics (Abs), ampicillin and gentamicin, were introduced into TMR-Lipo (denoted as TMR-Lipo-Abs) (FIG. 5A). TMR-Lipo-Abs were spherical particles with a hydrodynamic size of approximately 150.90 nm and a PDI value of 0.232 (FIG. 5B), which were not significantly different from those of TMR-Lipo (FIGS. 3B and 3C), and these results suggest that the loading of antibiotics into TMR-Lipo did not affect the physical properties.

It was also confirmed that TMR-Lipo-Abs had almost no impact on the viability of BMDM cells (FIG. 5C). Based on their safety and immunomodulatory activity, the therapeutic efficacy of TMR-Lipo-Abs was evaluated using a CLP-induced polymicrobial sepsis model. CLP modeling in mice was conducted to induce mid- or high-grade sepsis, including the severity grade of sepsis that is highly dependent on the position of cecal ligation.

As shown in FIG. 5D, in the mid-grade CLP, the mice treated with antibiotics alone exhibited approximately 50% mortality on day 2 post-CLP surgery, which was lower than that in the TMR-Lipo-treated group (mortality >75% on day 8). However, the TMR-Lipo-Abs group, possessing both antimicrobial and immunomodulation capabilities, exhibited dose-dependent protection. 80% of the mice were protected from CLP-induced mortality after TMR-Lipo-Abs was intraperitoneally injected at a dose of 2 mg/kg per mouse. In the high-grade CLP, treatment with antibiotics alone yielded survival benefits comparable to the CLP-PBS group, and deaths in both the PBS and antibiotic groups were concentrated at 24 to 36 h post-CLP, which may be due to hyperinflammatory responses in early-stage sepsis. Treatment with TMR-Lipo significantly improved survival rates compared to treatment with antibiotics alone, and the combination of the TMR peptide and antibiotics significantly reduced acute-phase mortality (FIG. 5E). Similarly, subcutaneous injection of TMR-Lipo into the CLP mice at two time points (1 h and 12 h) protected these mice from CLP-induced death (FIG. 5F). These results demonstrate that the TMR peptide is capable of controlling acute inflammation.

Consistent with the mortality data, serum concentrations of the key inflammatory cytokines, including TNF-α, IL-6, and IL-12p40, were significantly reduced in the TMR-Lipo-Abs group, whereas there was no change in the level of IL-10 after treatment with TMR-Lipo-Abs (FIG. 5G). In addition, histopathological analysis showed that treatment using TMR-Lipo-Abs significantly reduced the infiltration of immune cells and damage to the lungs, spleen, and liver compared to the control septic mice (FIG. 5I).

HMGB1 and PTX3 serve as proinflammatory cytokines that propagate inflammation responses through TLR4/RAGE binding, which is also a representative DAMP molecule indicating cell damage. As shown in FIG. 5H, TMR-Lipo-Abs decreased the expression of HMGB1 and PTX3 in the spleen, lungs, and liver of mice with sepsis. In summary, the histological and molecular analyses demonstrated that the synergistic combination of the TMR peptide and antibiotics mitigated excessive inflammation and prevented organ damage, thereby improving the survival benefit of patients with sepsis.

When the fusion peptide or TLR4/MD2/RAGE-targeting peptide (TMR peptide) according to the present invention is administered to a patient with sepsis, the inflammatory response can be reduced and/or sepsis can be treated or alleviated by inhibiting TLR4 and RAGE-mediated signaling.

For example, for purposes of claim construction, the claims set forth below should not be construed narrowly in any way compared to the literal language thereof, and thus exemplary embodiments from the specification should not be read as claims. Therefore, it should be understood that the present invention has been described by way of example, and is not a limitation on the scope of the claims. Therefore, the present invention is limited only by the claims below. All publications, issued patents, patent applications, books, and journal articles cited in the present application are hereby incorporated by reference in their entirety.

1. A fusion peptide in which four peptides consisting of amino acid sequences represented by SEQ ID NO: 2 to SEQ ID NO: 5 are connected by a linker.

2. The fusion peptide of claim 1, wherein the linker is (G)ₙ, (A)ₙ, or (G₄S)ₘ, wherein m is an integer from 1 to 3, and n is an integer from 2 to 8.

3. The fusion peptide of claim 1, comprising an amino acid sequence represented by (SEQ ID NO: 2)-(linker)-(SEQ ID NO: 3)-(linker)-(SEQ ID NO: 4)-(linker)-(SEQ ID NO: 5).

4. The fusion peptide of claim 1, comprising an amino acid sequence represented by SEQ ID NO: 1.

5. A pharmaceutical composition for treating sepsis, comprising the fusion peptide of claim 1.

6. The pharmaceutical composition of claim 5, wherein the fusion peptide is encapsulated in a liposome.

7. The pharmaceutical composition of claim 6, wherein the fusion peptide is bound to a membrane of the liposome.

8. A nucleic acid molecule encoding the fusion peptide of claim 1.

9. A recombinant vector comprising the nucleic acid molecule of claim 8.

10. A recombinant strain comprising the nucleic acid molecule of claim 8.

11. A health functional food for preventing or alleviating sepsis, comprising the fusion peptide of claim 1.
